(19) United States
(12) Patent Application Publication
(10) Pub. No.: US 2016/0075995 A1
(43) Pub. Date: Mar. 17, 2016

(54) SWEAT GLAND-DERIVED STEM CELLS AND METHODS OF USE

(71) Applicant: Krzysztof Kobielak, Los Angeles, CA (US)

(72) Inventor: Krzysztof Kobielak, Los Angeles, CA (US)

(21) Appl. No.: 14/856,424

(22) Filed: Sep. 16, 2015

Related U.S. Application Data
(60) Provisional application No. 62/051,422, filed on Sep. 17, 2014.

(51) Int. Cl. C12N 5/071; G01N 33/50; A61K 38/18; A61K 35/36; A61K 35/33

(52) U.S. Cl. CPC C12N 5/0633; A61K 35/36; A61K 35/33; A61K 38/1875; G01N 33/5073; G01N 33/502; C12N 2502/13

(57) ABSTRACT

Provided herein is a method to isolate a sweat gland stem cell and novel compositions containing the cell. Also provided are compositions and methods to clonally expand the population and differentiate the cells into various phenotypes. Therapeutic methods for the compositions are further provided.

### CROSS-REFERENCE TO RELATED APPLICATION

This application claims priority under 35 U.S.C. §119(e) to U.S. Provisional Application No. 62/051,422, filed Sep. 17, 2014, the content of which is hereby incorporated by reference in its entirety.

### BACKGROUND

Throughout this disclosure, various technical and patent publications are referenced to more fully describe the state of the art to which this invention pertains. Some of the references are identified by first author name and date of publication. These publications are incorporated by reference, in their entirety, into this application.

The identification of expandable, self-renewable stem cells isolated from sweat glands and the establishment of a protocol for culturing and grafting them will pave the way for practical cell-based therapy for wound healing, and in particular, tissue grafts for the treatment of burns and tissue reconstitution.

### SUMMARY

Slow cycling is a common feature shared among several stem cells (SCs) identified in adult tissues including hair follicle and cornea. Recently, existence of unipotent SCs in basal and lumenal layers of sweat gland (SG) has been described and label retaining cells (LRCs) have also been localized in SGs, however, whether these LRCs possess SCs characteristic has not been investigated further.

Here, Applicant used a H2BGFP LRCs system for in vivo detection of infrequently dividing cells. This system allowed Applicant to specifically localize and isolate SCs with label-retention and myoepithelial characteristics restricted to the SG proximal acinar region. Using an alternative genetic approach, Applicant demonstrated that SG LRCs expressed keratin 15 (K15) in the acinar region and lineage tracing determined that K15 labeled cells contributed long term to the SG structure but not to epidermal homeostasis. Surprisingly, wound healing experiments did not activate proximal acinar SG cells to participate in epidermal healing. Instead, predominantly non-LRCs in the SG duct actively divided, whereas the majority of SG LRCs remained quiescent. However, when Applicant further challenged the system under more favorable isolated wound healing conditions, Applicant was able to trigger normally quiescent acinar LRCs to trans-differentiate into the epidermis and adopt its long term fate. In addition, dissociated SG cells were able to regenerate SGs and, surprisingly, hair follicles demonstrating their in vivo plasticity. By determining the gene expression profile of isolated SG LRCs and non-LRCs in vivo, Applicant identified several Bone Morphogenetic Protein (BMP) pathway genes to be up-regulated and confirmed a functional requirement for BMP receptor 1A (BMPR1A)-mediated signaling in SG formation.

This data highlight the existence of SG stem cells (SGSCs) and their primary importance in SG homeostasis. It also emphasizes SGSCs as an alternative source of cells in wound healing and their plasticity for regenerating different skin appendages.

In view of the above, the present disclosure provides an isolated self-renewable sweat gland (“SG”) stem cell and a clonal population of the stem cell that are useful in such therapies. The self-renewable sweat gland stem cell or clone is multipotent can be isolated from the proximal acinar gland region and not in the SG ductal region of mammalian sweat glands. In some embodiments, these multipotent stem cells are capable of differentiating into at least one, or alternatively at least two, or alternatively at least three, of sweat glands, hair follicles and epidermis, making them particularly useful for skin grafts.

Also provided is an isolated population of self-renewable multipotent sweat gland stem cells. In some embodiments, the isolated population of self-renewable multipotent sweat gland stem cells is substantially homogenous, i.e., greater than 60%, or alternatively greater than 70%, or alternatively greater than 80%, or alternatively greater than 85%, or alternatively greater than 90%, or alternatively greater than 95%, of the multipotent sweat gland stem cells.

Methods of isolating, preparing, culturing, expanding, propagating and/or differentiating the stem cells, and methods of using the cells or populations for treatment are also disclosed in the current disclosure.

### BRIEF DESCRIPTION OF THE DRAWINGS AND TABLES

FIG. 1 shows sweat gland LRCs are localized in the acinar gland region of SGs. (Panels A, B) H2BGFP is expressed in the epidermis and sweat glands before doxycycline treatment. (Panels C, D) Sweat gland LRCs are found in the acinar gland region after 4 weeks of chase with doxycycline. (Panels E, F) Tissue histology on sections with H&E staining Abbreviations: LRCs, label-retaining cells; H2BGFP, histone 2B conjugated with green fluorescent protein; H&E, hematoxylin and eosin staining.

FIG. 2 shows sweat gland LRCs are attached to the basement membrane and possess myoepithelial characteristics. (Panel A) Sweat gland LRCs are attached to the basement membrane positive for P4 integrin (red) and are (Panel B) found in the basal layer co-localizing with K14. (Panels C, D) Sweat gland LRCs do not express lumenal layer markers K8 and K18, respectively. (Panel E) Within the basal layer, LRCs co-localize with myoepithelial cell marker p63, inset denotes p63 single channel, arrows and (Panel F) with myoepithelial cell marker SMA, arrows. (Panels G, H) Whole mount staining of SG LRCs with laminin with and without DAPI. Arrows denote co-localization of markers with H2BGFP marked LRCs. Abbreviations: LRCs, label-retaining cells; H2BGFP, histone 2B conjugated with green fluorescent protein; SMA, smooth muscle actin; K, keratin.

FIG. 3 shows sweat gland LRCs express Keratin 15 and contribute long term to the acinar SG structure. (Panel A) K15 staining of sweat gland LRCs indicate positive K15 expression. (Panel B) Fluorescent photo of K15CrePR/R26eYFP^{RU} palm containing YFP positive sweat glands after long term YFP activation. (Panel C) Section of K15CrePR/R26tdTom^{RU} crossed onto KSTetOff/TreH2BGFP sweat glands after 4 weeks of chase with doxycycline followed by 2 days of RU treatment. (Panel D) K14 basal layer staining co-localizes with GFP expression in K15-GFP transgenic sweat glands. (Panel E) K18 lumenal marker staining co-localizes with K15-GFP expression in sweat glands. (Panel F) FACS analysis of K15-GFP sweat glands demonstrates that approximately half of the K15-GFP positive cells are localized to the basal layer expressing α6 integrin. (Panel G) Histology of X-Gal-treated K15CrePR/R26LacZ transgenic mice, blue stain indicates transgene expression for more than 6 months after RU activation in sweat glands. (Panel H) K15 expression co-localizes with LRCs in the acinar sweat gland region.

FIG. 4 shows isolation strategy for sweat gland LRCs. (Panel A) Whole KSTetOff/TreH2BGFP toe tip under the stereomicroscope, dotted yellow line marks sweat gland dissection. (Panel B) Dissected sweat glands with surrounding sole's epidermis. (Panel C) Separation of sweat glands from attached sole's epidermis after collagenase treatment. (Panel D) A series of further enzymatic digestions result in a single cells suspension of sweat gland cells for FACS. (Panel E) Sweat gland cells sorted for H2BGFP and α6 integrin-PE double positive cells as well as α6 integrin-PE single positive surrounding basal cells.

FIG. 5 shows molecular characteristics of sweat gland LRCs define BMP signaling as a requirement for SG formation. (Panel A) GFP+/α6+ sweat gland LRCs and GFP−/α6+ sweat gland non-LRCs basal cells were compared to the basal layer of the sole's epidermis. The resulting gene expression profiles of these sweat gland LRCs and basal cells were then compared to each other. (Panel B) Gene expression profiles consistently found in two independent microarray analyses from independent biological samples were categorized into ion and protein transport, signaling, transcription, extracellular matrix (ECM) and cell adhesion based on function. (Panel C) Sodium Potassium ATPases were confirmed to be expressed in the sweat glands. (Panel D) Gja1 is confirmed to be expressed in SG LRCs (arrows) as well as non-LRCs. (Panel E) Phospho-Smad2 co-localizes with SG LRCs, arrows. (Panel F) Corresponding phospho-Smad2 and K8 channels. Arrows indicate LRCs marked in panel “E”. (Panel G) Positive phospho-smad1/5/8 staining indicates active BMP signaling in sweat glands. (Panel H) Corresponding phospho-Smad1/5/8 and K8 channels with arrows indicating co-localization with some LRCs. (Panel I) Downgrowth of sweat glands is observed in P1 control paws but is (Panel J) absent in Bmpr1a/K14Cre/K14-H2BGFP KO paws. (Panel K) Similarly, more developed sweat glands are observed in P8 control paws but are (Panel L) still absent in KO mice. (Panel M) The basal layer of the epidermis as well as the sweat glands is proliferative at P1. (Panel N) Although sweat glands are absent in Bmpr1a/K14Cre/K14-H2BGFP KO paws, the epidermal basal layer is still capable of division.

FIG. 6 shows acinar sweat gland cells do not contribute to the epidermis during typical wound healing. (Panel A) K15CrePR/R26LacZRU marked sweat gland cells do not contribute to the epidermis at 24 h, (Panel B) 48 h, and (Panel C) 72 h after wounding. (Panel D) BrdU pulse shows that a few SG cells are activated upon injury (inset, arrows) while most SG LRCs remain quiescent. (Panel E) Ki67 staining confirms that the acinar sweat gland region is quiescent while the SG duct and epidermal basal layer is proliferative at 24 h and (Panel F) 48 h. (Panel G) Under normal homeostasis, cells of the SG duct and epidermal basal layer are active in the cell cycle. (Panel H) Corresponding single Ki67 (red) channel. Abbreviations: du, ducts.

FIG. 7 shows sweat gland LRCs can trans-differentiate into the epidermis under prolonged isolated wound healing conditions. (Panel A) DIC photo of the back skin area containing transplanted H2BGFP labeled sweat glands at 34 days. (Panel B) Corresponding fluorescent image displaying the presence of H2BGFP positive cells in the grafted area. Arrows indicate regions with positive H2BGFP cells while “*” marks autofluorescence of the wounded area. (Panel C) Biopsy was taken at 38 days where H2BGFP sweat glands were observed in the dermis, arrow. (Panel D) Higher exposure time for the GFP channel on the same section from (Panel C) detected marked cells with lower H2BGFP intensities in the epidermis. (Panels E, I) K5 basal layer staining demonstrating the contribution of sweat gland cells to the newly formed epidermis at 38 and 46 days, respectively. (Panels F, J) These H2BGFP labeled cells are still able to proliferate as indicated by Ki67 staining (Panels G, K) Sweat gland cells can differentiate into cells of the suprabasal layer marked by K1 at 38 and 46 days, respectively. (Panels H, L) These sweat gland cells can also contribute to the granular layer as marked by loricrin. (Panels M, N) When 4 weeks chased sweat glands are transplanted and kept on doxycycline treatment, the H2BGFP label gets diluted out of some sweat glands (arrows), as confirmed by K5 and K8 staining for sweat glands. (Panel 0) Ki67 staining show that these sweat glands lacking visible H2BGFP LRCs (green—nuclear) defined by K5 positive staining (green membrane staining) contains Ki67 positive dividing cells (arrows). (Panel P) H2BGFP sweat gland LRCs can contribute to the newly formed epidermis as indicated by K5 basal layer staining (arrows). (Panel Q) Some sweat glands are connected to the newly formed epidermis lacking visible H2BGFP LRCs. (Panel R) Sweat gland LRCs contributing to the epidermis are proliferative near the CD104 marked epidermal basal layer as marked by Ki67, inset shows magnification of co-localization. (Panel S) Sweat gland LRCs can contribute to the suprabasal layer marked by K1 as well as the (Panel T) granular layer expressing loricrin. White dotted lines mark dermal-epidermal interfaces. Abbreviations: CD104, P4 integrin.

FIG. 8 shows dissociated sweat gland cells can regenerate sweat glands, hair follicles, and the epidermis. (Panel A) Chamber graft of sweat glands dissociated into a single cell suspension labeled with H2BGFP (chased for 4 weeks) mixed with newborn unmarked dermal fibroblasts yields GFP positive hair-like fibers at 29 days. (Panel B) Section through graft confirms the presence of H2BGFP positive hair follicles. (Panel C) K5 staining marks the outer root sheath of this H2BGFP+ hair follicle. (Panel C′) AE15 stains the inner root sheath and medulla, arrows. (Panel C″) AE13 stains the hair shaft. (Panel D) 70 days after transplantation, H2BGFP positive sweat gland structures were found, as confirmed by Na⁺/K⁺ ATPase expression (inset), in addition to H2BGFP positive hair follicle. (Panel E) Magnification of the hair follicle in (Panel D) with (Panel E′) GFP single channel. (Panel F) The sweat gland structures found also expressed K5 basal and K8 lumenal layer markers. (Panel G) H2BGFP positive cells were also found in the K5 basal layer, (Panel H) K1 suprabasal layer, and (Panel I) loricrin marked granular layer of the newly regenerated epidermis. (Panels J, K) In an independent experiment, 39 days after subcutaneous injections of 4 weeks chased unsorted dissociated cells from H2BGFP labeled SGs with unmarked newborn dermal fibroblasts, a cluster of GFP positive cells containing hair follicles were observed. (Panel L) Magnification of a GFP+ hair follicle from panel (Panel K). (Panel M) Sections show the presence of H2BGFP labeled SG structures expressing K8 lumenal layer marker. (Panel N) H2BGFP positive cells are again also found in the basal, suprabasal, and (Panel 0) granular layers of the epidermis. White dotted lines mark dermal-epidermal interfaces.

FIG. 9 shows sweat gland LRCs possess slow cell cycle dynamics but are non post-mitotic cells. Hair follicle bulge at (Panel A) 4 weeks, (Panel B) 10 weeks, (Panel C) 15 weeks and (Panel D) 20 weeks of chase with doxycycline. 10× magnification of sweat glands at (Panel E) 4 weeks, (Panel F) 10 weeks, (Panel G) 15 weeks and (Panel H) 20 weeks of chase with doxycycline. 20× magnification of sweat glands at (Panel I) 4 weeks, (Panel J) 10 weeks, (Panel K) 15 weeks and (Panel L) 20 weeks of chase with doxycycline. Exposure time of all 10× images is 800 ms and 20× images is 160 ms. White dotted lines mark dermal-epidermal interfaces.

FIG. 10 shows validation of genes identified in the microarray analysis by real time PCR. Using SG LRCs as the baseline, Applicant confirmed an up-regulation of Bgn, Mmp2, and Timp2 in SG non-LRCs (α6+ basal layer cells) when compared to GFP+/α6+SG LRCs in either 2 or 3 independent biological samples. Representative data from one is shown. Error bars represent standard deviation.

Table 1 shows common DEG list for both SG LRCs and SG non-LRCs. Functionally categorized list of genes commonly identified in both SG LRCs (GFP+/α6+) and SG non-LRCs (GFP−/α6+) when compared to the basal layer of the sole's epidermis.

Table 2 shows unique DEG list for SG LRCs. Functionally categorized list of genes identified in SG LRCs (GFP+/α6+) when compared to the basal layer of the sole's epidermis.

Table 3 shows unique DEG list for SG non-LRCs. Functionally categorized list of genes identified in the basal layer SG non-LRCs (GFP−/α6+) when compared to the basal layer of the sole's epidermis.

### DETAILED DESCRIPTION

### Definitions

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

The practice of the present invention will employ, unless otherwise indicated, conventional techniques of tissue culture, immunology, molecular biology, microbiology, cell biology and recombinant DNA, which are within the skill of the art. See, e.g., Sambrook, Fritsch and Maniatis (1989) Molecular Cloning: A Laboratory Manual, 2^{nd} edition; F. M. Ausubel, et al. eds. (1987) Current Protocols In Molecular Biology; the series Methods in Enzymology (Academic Press, Inc.): PCR 2: A Practical Approach (1995) (M. J. MacPherson, B. D. Hames and G. R. Taylor eds.); Harlow and Lane, eds. (1988) Antibodies, A Laboratory Manual; Harlow and Lane, eds. (1999) Using Antibodies, a Laboratory Manual; and R. I. Freshney, ed. (1987) Animal Cell Culture.

All numerical designations, e.g., pH, temperature, time, concentration, and molecular weight, including ranges, are approximations which are varied (+) or (−) by increments of 1.0 or 0.1, as appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a,” “an” and “the” include plural references unless the context clearly dictates otherwise.

As used herein, the term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of” shall mean excluding more than trace elements of other ingredients and substantial method steps. Embodiments defined by each of these transition terms are within the scope of this invention.

The term “isolated” means separated from constituents, cellular and otherwise, in which the cell, tissue, polynucleotide, peptide, polypeptide, protein, antibody or fragment(s) thereof, which are normally associated in nature. For example, an isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart. An isolated cell is a cell that is separated form tissue or cells of dissimilar phenotype or genotype. In one aspect, the isolated naturally occurring stem cell or other composition is combined with an element with which it is not normally found in nature. Non-limiting examples of such include, detectable labels, polynucleotides, proteins or peptides and in combination with other compositions such as carriers, e.g., pharmaceutically acceptable carriers and supports and growth factors.

As used herein, “stem cell” defines a cell with the ability to divide (and self-renewal) for indefinite periods in culture and/or long-term contribution in vivo in tissue during normal homeostasis as well as reconstitution and give rise to specialized cells (fates) in tissue specific differentiation. At this time and for convenience, stem cells are categorized as somatic (adult), embryonic or induced pluripotent stem cells. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. Non-limiting examples of embryonic stem cells are the HES2 (also known as ES02) cell line available from ESI, Singapore and the H1 or H9 (also known as WA01) cell line available from WiCell, Madison, Wis. Additional lines are pending NIH review. See for examplegrants.nih.gov/stem_cells/registry/current.htm (last accessed Oct. 2, 2009). Pluripotent embryonic stem cells can be distinguished from other types of cells by the use of markers including, but not limited to, Oct-4, alkaline phosphatase, CD30, TDGF-1, GCTM-2, Genesis, Germ cell nuclear factor, SSEA1, SSEA3, and SSEA4. An induced pluripotent stem cell (iPSC) is an artificially derived stem cell from a non-pluripotent cell, typically an adult somatic cell, produced by inducing expression of one or more stem cell specific genes.

The term “propagate” means to grow or alter the phenotype of a cell or population of cells. The term “growing” refers to the proliferation of cells in the presence of supporting media, nutrients, growth factors, support cells, or any chemical or biological compound necessary for obtaining the desired number of cells or cell type. In one embodiment, the growing of cells results in the regeneration of tissue. In yet another embodiment, the tissue is comprised of neuronal progenitor cells or neuronal cells.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells.

As used herein and as set forth in more detail below, “conditioned medium” is medium which was cultured with a mature cell that provides cellular factors to the medium such as cytokines, growth factors, hormones, and extracellular matrix.

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell. As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

Examples of cells that differentiate into ectodermal lineage include, but are not limited to epidermal cells, neurogenic cells, and neurogliagenic cells.

As used herein, the term “differentiates or differentiated” defines a cell that takes on a more committed (“differentiated”) position within the lineage of a cell. “Dedifferentiated” defines a cell that reverts to a less committed position within the lineage of a cell.

As used herein, a “multipotent cell” defines a less differentiated cell that can give rise to at least two distinct (genotypically and/or phenotypically) further differentiated progeny cells. In another aspect, a “pluripotent cell” includes an induced Pluripotent Stem Cell (iPSC) which is an artificially derived stem cell from a non-pluripotent cell, typically an adult somatic cell, produced by inducing expression of one or more stem cell specific genes. Such stem cell specific genes include, but are not limited to, the family of octamer transcription factors, i.e., Oct-3/4; the family of Sox genes, i.e., Sox1, Sox2, Sox3, Sox 15 and Sox 18; the family of Klf genes, i.e. Klf1, Klf2, Klf4 and Klf5; the family of Myc genes, i.e., c-myc and L-myc; the family of Nanog genes, i.e. OCT4, NANOG and REX1; or LIN28. Examples of iPSCs are described in Takahashi et al. (2007) Cell advance online publication 20 Nov. 2007; Takahashi & Yamanaka (2006) Cell 126:663-76; Okita et al. (2007) Nature 448:260-262; Yu et al. (2007) Science advance online publication 20 Nov. 2007; and Nakagawa et al. (2007) Nat. Biotechnol. Advance online publication 30 Nov. 2007.

“Self-renewable” refers to a cell being able to self-renew for over a number of passages without substantial changes of cell properties. In one aspect, the number of passages is at least about 5, or alternatively at least 10, or alternatively at least about 15, 20, 30, 50, or 100.

As used herein, the “lineage” of a cell defines the heredity of the cell, i.e. its predecessors and progeny. The lineage of a cell places the cell within a hereditary scheme of development and differentiation.

As used herein, the term “differentiates or differentiated” defines a cell that takes on a more committed (“differentiated”) position within the lineage of a cell. “Dedifferentiated” defines a cell that reverts to a less committed position within the lineage of a cell.

Clonal and subclonal population of cells are cells that maintain the original phenotypic markers and multipotency as the parent cell from which is was reproduced.

A “clonal culture” is a group of cells originated from one ancestor cell. Subclonal culture is a group of cells originated from one of clonally cultured cell. By comparing parental clonal and descendant subclonal culture, one should be able to determine whether subclonal population maintain the original phenotypic markers and multipotency.

“Bone Morphogenic Proteins” (BMP) are a group of multifunctional growth factors and cytokines with effects in various tissues. For example, BMPs are known to induce the formation of bone and/or cartilage. Examples of BMP may include, but are not limited to BMP1, BMP2, BMP3, BMP4, BMP5, BMP6, BMP7, BMP8a, BMP8b, BMP10 and BMP15.

“BMP signaling” or “BMP signaling pathway” refers to the enzyme linked receptor protein signaling transduction pathway involving proteins that directly or indirectly regulate (activate or inhibit) downstream protein activity or gene expression. Examples of molecules involved in the BMP signaling pathways may be found in the public Gene Ontology (GO) database, under GO ID: GO:0030509, accessible at the web page (amigo.geneontology.org/cgi-bin/amigo/term-details.cgi?term=G0:0030509&session id=5573amigo1226631957), last accessed on Nov. 17, 2008. Without limitation, examples of proteins in the BMP signaling pathway include Activin receptor type-1 (ACVR1, UniProt: Q04771), Activin receptor type-2A (ACVR2A, UniProt: P27037), Activin receptor type-2B (ACVR2B, UniProt: Q13705), BMP1 (UniProt: P13497), BMP2 (UniProt: P12643), BMP3 (UniProt: P12645), BMP4 (UniProt: P12644), BMP5 (UniProt: P22003), BMP6 (UniProt: P22004), BMP7 (UniProt: P18075), BMP8a (UniProt: Q7Z5Y6), BMP8b (UniProt: P34820), BMP10 (UniProt: 095393), BMP15 (UniProt: 095972), Bone morphogenetic protein receptor type-1A (BMPR1A, UniProt: P36894), Bone morphogenetic protein receptor type-1B (BMPR1B, UniProt: 000238), Bone morphogenetic protein receptor type-2 (BMPR2, UniProt: Q13873), Chordin-like protein (CHRDL1, UniProt: Q9BU40), Follistatin-related protein 1 (FSTL1, UniProt: Q12841), Growth/differentiation factor 2 (GDF2, UniProt: Q9UK05), Growth/differentiation factor 6 (GDF6, UniProt: Q6KF10), Growth/differentiation factor 7 (GDF7, UniProt: Q7Z4P5), Gremlin-2 (GREM2, UniProt: Q9H772), RGM domain family member B (RGMB, UniProt: Q6NW40), Ski oncogene (SKI, UniProt: P12755), Mothers against decapentaplegic homolog 4 (SMAD4, UniProt: Q13485), Mothers against decapentaplegic homolog 5 (SMAD5, UniProt: Q99717), Mothers against decapentaplegic homolog 6 (SMAD6, UniProt: 043541), Mothers against decapentaplegic homolog 7 (SMAD7, UniProt: 015105), Mothers against decapentaplegic homolog 9 (SMAD9, UniProt: 015198), E3 ubiquitin-protein ligase SMRF2 (SMURF2, UniProt: Q9HAU4), TGF-beta receptor type III (TGFBR3, UniProt: Q03167), Ubiquitin-conjugating enzyme E2 D1 (UBE2D1, UniProt: P51668), Ubiquitin-conjugating enzyme E2 D3 (UBE2D3, UniProt: P61077) and Zinc finger FYVE domain-containing protein 16 (ZFYVE16, UniProt: Q7Z3T8). Proteins that positively or negatively regulate the BMP signaling, for purpose of this invention, are also considered within the meaning of the BMP signaling. Proteins that positively regulate BMP signaling include, but are not limited to, Serine/threonine-protein kinase receptor R3 (ACVRL1, UniProt: P37023) and Endoglin (ENG, UniProt: P17813). Proteins that negatively regulate BMP signaling include, but are not limited to, Chordin (CHRD, UniProt: Q9H2X0), E3 ubiquitin-protein ligase SMURF1 (SMURF1, UniProt: Q9HCE7), Sclerostin (SOST, UniProt: Q9BQB4) and Brorin (VWC2, UniProt: Q2TAL6). Examples of proteins in the BMP signaling pathway may also include Proprotein convertase subtilisin/kexin type 6 (PCSK6, UniProt: P29122) that regulates BMP signaling.

Small molecules, polynucleotides, polypeptides that enhance or inhibit BMP signaling exist or can be made with procedures known by those skilled in the art. Yanagita (2009) BioFactors 35(2):113-199 is a review article discussing BMP regulators (incorporated by reference). For example, dorsomorphin is a potent small molecule BMP antagonist (Hao et al. (2008) PLoS ONE 3(8):e2904, Yu et al. (2008) Nat Chem Biol. 4(1):33-41). Dorsomorphin is currently commercially available from several vendors. Dorsomorphin was reported to selectively inhibit the BMP receptors, type I: ALK2, ALK3 and ALK6 and thus “blocks BMP-mediated SMAD1/5/8 phosphorylation”. Dorsomorphin has preferential specificity toward inhibiting BMP versus TGF-beta and activin signaling. In published reports, dorsomorphin is characterized by low toxicity. It can be delivered into skin to lower macro-environmental BMP signaling and create favorable conditions for hair growth to occur. A unique property of dorsomorphin is that it is a small molecule and is soluble in DMSO. DMSO is known to significantly facilitate trans-dermal delivery of small molecule drugs. This enhancing effect of DMSO on skin penetration can be used in non-invasive method of pharmacological modulation of dermal macro-environment. Treatment procedure thus consists of simply applying liquid form of dorsomorphin in DMSO onto the surface of intact skin. Dorsomorphin in DMSO can be made in form of cream that can be simply rubbed onto intact skin. Small molecule agonist and antagonists for other signaling pathways also exist and can be used to augment or inhibit BMP signaling. Interaction of these small molecules with pathways including, but not limited to, WNT, SHH and FGF will also have direct or indirect impact on BMP signaling thus serve as effective modulator of hair growth via methods disclosed in this invention.

In some aspects, an agent that can augment or inhibit BMP signaling is a small molecule agonist or antagonist to a BMP agonist or antagonist. In one aspect, the small molecule is a noggin agonist. In another aspect, the small molecule is a noggin antagonist.

Examples of agents that can augment or inhibit BMP signaling also include, but are not limited to, polynucleotides that encode BMP proteins, encode polypeptides augmenting or inhibiting BMP signaling, or augmenting or inhibit expression of BMP proteins, or polypeptides augmenting or inhibiting BMP signaling. In some embodiments, the agent is small interference RNA (siRNA) or double strand RNA (dsRNA) that inhibits expression of proteins that augment or inhibit BMP signaling.

Examples of agents that can augment or inhibit BMP signaling may also include, but are not limited to, an isolated or recombinant BMP protein, or isolated or recombinant polypeptide enhancing or inhibiting BMP signaling. In some aspect, the agent further comprises a pharmaceutically acceptable carrier. In another aspect, the compositions contain carriers that modulate (controlled release) the release of the active agent when administered to a subject in need thereof.

Examples of polypeptide agents that augment or inhibit BMP signaling may also include, but are not limited to, antibodies or modified antibodies including, but not limited to, blocking fragments of antibodies, that activate, stabilize or inhibit proteins in the BMP signaling pathway or proteins modulating the BMP signaling pathway, thereby augmenting or inhibiting BMP signaling.

As used herein, the term “modulate” refers to an act by an agent to regulate, to control or to change certain characteristics of the formation of pilosebaceous units. Examples of the agent may include, but are not limited to, proteins or polypeptides, DNA, RNA, siRNA, dsRNA or other polynucleotides, small molecules. The agent may also mean a temperature change, physical movement or stimulus or any other therapeutic or clinical means that alter the formation of pilosebaceous units. Without limitation, the object may mean a biochemical molecule or pathway, a biochemical activity, a medical condition or any other chemical, biochemical, physical or medical aspect of a subject. In one aspect, the term “modulate” means to enhance the formation of pilosebaceuous units in a plane. In another aspect, the term “modulate” means to inhibit the formation of pilosebaceous units on a plane.

A “composition” is also intended to encompass a combination of active agent and another carrier, e.g., compound or composition, inert (for example, a detectable agent or label) or active, such as an adjuvant, diluent, binder, stabilizer, buffers, salts, lipophilic solvents, preservative, adjuvant or the like. Carriers also include pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this invention, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively, more than 95%, of the cells are of the same or similar phenotype. Phenotype can be determined by a pre-selected cell surface marker or other marker.

“Detectable labels” or “markers” include, but are not limited to radioisotopes, fluorochromes, chemiluminescent compounds, dyes, and proteins, including enzymes. Non-limiting examples of such include a gene encoding an enhanced green fluorescent protein (EGFP), red flouresence protein (RFP), green fluorescent protein (GFP) and yellow fluorescent protein (YFP) or the like. These are commercially available and described in the technical art.

Sweat gland stem cell (SGSC) are a multipotent cell type that can generate a variety of cell types, including sweat glands, hair follicles and the epidermis. The cells can be identified by a series of markers which include but are not limited to genes important for Bone Morphogenetic Protein (BMP) signaling, including Bmpr1, Bmpr2, Smad5, id2 id3 and decorin that are shown to be upregulated in the SGSC when compared to the epidermis. Marker analysis of the SGSC is provided in the Example and incorporated herein by reference.

The SGSC can also be identified by its multipotency, e.g., the capacity to differentiate into at least one tissue or cell type selected from the group of a sweat gland, hair follicle and epidermis.

“Treating” or “treatment” of a disease includes: (1) preventing the disease, i.e., causing the clinical symptoms of the disease not to develop in a patient that may be predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

The term “effective amount” refers to a concentration or amount of a reagent or composition, such as a composition as described herein, cell population or other agent, that is effective for producing an intended result, including cell growth and/or differentiation in vitro or in vivo, or for tissue regeneration. It will be appreciated that the number of cells to be administered will vary depending on the specifics of the disorder to be treated, including but not limited to size or total volume/surface area to be treated, as well as proximity of the site of administration to the location of the region to be treated, among other factors familiar to the medicinal biologist and/or treating physician.

The terms effective period (or time) and effective conditions refer to a period of time or other controllable conditions (e.g., temperature, humidity for in vitro methods), necessary or preferred for an agent or composition to achieve its intended result, e.g., the differentiation of cells to a pre-determined cell type.

The term patient or subject refers to animals, including mammals, such as murine, canine, feline, equine, bovine, simian or humans, who are treated with the pharmaceutical compositions or in accordance with the methods described herein.

The term pharmaceutically acceptable carrier (or medium), which may be used interchangeably with the term biologically compatible carrier or medium, refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Pharmaceutically acceptable carriers suitable for use in the present invention include liquids, semi-solid (e.g., gels) and solid materials (e.g., cell scaffolds and matrices, tubes sheets and other such materials as known in the art and described in greater detail herein). These semi-solid and solid materials may be designed to resist degradation within the body (non-biodegradable) or they may be designed to degrade within the body (biodegradable, bioerodable). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways.

The term administration shall include without limitation, administration by oral, topical. parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The disclosue is not limited by the route of administration, the formulation or dosing schedule.

The terms autologous transfer, autologous transplantation, autograft and the like refer to treatments wherein the cell donor is also the recipient of the cell replacement therapy. The terms allogeneic transfer, allogeneic transplantation, allograft and the like refer to treatments wherein the cell donor is of the same species as the recipient of the cell replacement therapy, but is not the same individual. A cell transfer in which the donor's cells and have been histocompatibly matched with a recipient is sometimes referred to as a syngeneic transfer. The terms xenogeneic transfer, xenogeneic transplantation, xenograft and the like refer to treatments wherein the cell donor is of a different species than the recipient of the cell replacement therapy.

### Detailed Embodiments

In one aspect, this invention provides an isolated self-renewable sweat gland stem cell (SGSC). The isolated self-renewable SGSC stem cell can be isolated from any source, examples of which include without limitation, any animal (alive or dead) so long as the tissue containing the SGSC is viable. Thus, the isolated SGSC can be animal, e.g., mammalian such as equine, feline, canine, porcine, bovine, murine, simian, and human.

The SGSC is isolated from the tissue source by any means that allows for isolation of a single cell by use of an identifying marker, e.g., FACS analysis. Details of this procedure are provided herein.

In one aspect, the isolated SGSC are isolated using FACS analysis and the stem cell markers to isolate the cell and composition are provided herein.

In addition to the markers, the isolated cell is identifiable by its multipotency, e.g., it is capable of differentiation into at least one, two or all three cell or tissue type selected from sweat gland, hair follicle and epidermis. Confirmation of the differentiation state of the cells can be performed by identification of cell type specific markers as known to those of skill in the art and as identified herein. In one aspect, the isolated sweat gland stem cell is capable of differentiation into at least two of the cell types. In another aspect, the isolated sweat gland stem cell is capable of differentiation into at least two, or alternatively at three tissue or cell types

In a further aspect, this invention provides isolated clonal population or a population of substantially homogeneous, i.e., at least 50%, or alternatively at least 70%, or alternatively at least 80%, or alternatively at least 85%, or alternatively at least 90%, or alternatively at least 95%, of the isolated sweat gland stem cell as described above. The clonal population contains majorities of the characteristics of the isolated cell as identified above.

This disclosure also provides an isolated SGSC as described above or an isolated population of same further comprising an exogenous agent, e.g., a small molecule, detectable label (e.g., a label for use in FACs analysis), growth factor, differentiating factor, protein, polypeptide, fibroblast, antibody or a non-naturally occurring nucleic acid, e.g., a therapeutic nucleic acid. Thus, these compositions are useful in the therapeutic and diagnostic methods as described herein as well as the screens for new therapeutic agents. In one aspect, the cells and compositions are useful for the treatment of wound in a subject by administering and effective amount of the cells and/or compositions. Another aspect relates to a method for promoting wound healing, promoting or increasing wound healing, decreasing the size of a wound, or decreasing the time to wound healing in a subject patient in need thereof comprising administering the isolated cell or population of the isolated cell as described herein.

This disclosure also provides methods for isolating a SGSC and/or a method for preparing a substantially homogeneous population of isolated sweat gland stemcells or populations as described. To isolate the SGSC, the method requires contacting a source cell, population or tissue likely to contain the SGSC with a detectably labeled antibody or other ligand that is specific for one or more identifying marker as identified above. After sufficient time and under appropriate conditions to allow the ligand to bind the marker to form a ligand-marker complex. The cells having the ligand-marker complex are then separated by any appropriate means, e.g., by FACs, from those that do not have a ligand-marker complex, thereby preparing an isolated SGSC.

In a further aspect, this disclosure provides a method for preparing a clonal population, a mass culture and/or differentiating an isolated sweat gland stem cell as described above or the population as described above by contacting the cell or population with an effective amount of a clonal expansion medium or differentiation medium as described herein and culturing of the cells under the appropriate conditions to obtain any of a clonal population or a mass culture or yet further differentiation into a selected lineage. In one aspect, the method prepares an expanded substantially homogenous population of SGSCs, or sweat glands, epidermal cells, or hair follicle cells. These populations are useful in the therapeutic and diagnostic methods as described herein as well as the screens for new therapeutic agents. The contacting may be performed in vitro or in vivo, depending on the intended use. For example, the isolated cell or population of cell can be implanted (autologous or allogeneic) into a subject and appropriate conditions can be locally administered to induce expansion and/or differentiation. Alternatively, the microenvironment of the cells will induce the appropriate differentiation of the cells into the cells and tissue. Yet further, agents can be administered to the subject to induce local expression of the agents that in turn, induce expansion and differentiation.

The isolated cells and/or populations of cells as described herein can be further combined with carrier, e.g., a pharmaceutically acceptable carrier or biocompatible matrix, for ease of administration.

The cell compositions as described herein are useful therapeutically and diagnostically. The compositions comprise, or alternatively consisting essentially of, or yet consisting of an isolated population of stem cells and a carrier, that optionally comprise an agent that maintains pluripotency of the cells in composition and/or a preservative. In one aspect, this disclosure provides a method for treating a wound, a burn or tissue grafting or associated disease, disorder or condition as is apparent to those of skill in the art, in a subject in need thereof, comprising, alternatively consisting essentially of, or yet further consisting of administering to the subject an effective amount of the isolated sweat gland stem cell as described above or the population as describe herein thereby treating the SGSC treatable disease, disorder or condition. Methods of administering cell populations are well known in the art and will depend on the treatment and individual. One or more administrations may be necessary. The cells may be autologous, allogeneic syngeneic or xenogeneic to the subject being treated. The subjects can be mammalian, e.g., bovine, feline, canine, equine or a human patient.

This disclosure also provides the use of the isolated sweat gland stem cell or the population of as described herein in the manufacture of a medicament as well as compositions containing the same. In one aspect, the medicament is to treat a SGSC treatable disease, disorder or condition.

The invention provides an article of manufacture, comprising packaging material and at least one vial comprising a solution of at least one agent or composition with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further comprises an article of manufacture, comprising packaging material, a first vial comprising at least one agent or composition and a second vial comprising an aqueous diluent of prescribed buffer or preservative, wherein said packaging material comprises a label that instructs a patient to reconstitute the therapeutic in the aqueous diluent to form a solution that can be held over a period of twenty-four hours or greater.

A method for identifying an agent that modulates the growth or differentiation of the isolated sweat gland stem cell is further provided by this invention. The method comprises, or alternatively consisting essentially of, or yet further consisting of, using the isolated cell or the population of cells and contacting the cell or the population with the agent wherein a change of growth or differentiation of the cell or population indicates that the agent modulates the growth or differentiation of the cell or the population.

### Example

### Materials and Methods

### Immunohistochemistry and Immunofluorescence Staining

All frozen sections were fixed in 4% Paraformaldehyde. Tissue sections were stained with hematoxylin and eosin for H&E visualization. For LacZ visualization, frozen sections were fixed in 0.2% Glutaraldehyde for 1 min, washed with PBS, and stained with 1 mg/ml X-gal overnight at 37° C. For immunofluorescence staining, sections were permeabilized with 0.1% Triton X-100 for 10 min and blocked in 0.1% Triton-PBS, 0.5% goat serum, and 0.1% BSA for 1 h at room temperature. Primary antibodies were incubated in the blocking buffer overnight at 4° C., wash with PBS. Secondary antibodies were incubated in 0.1% BSA for 1 h at room temperature. The following primary antibodies were used: K14 (1:200; gift from E. Fuchs Lab), CD104 (1:100; BD Pharmingen, 553745), K8 (TROMA-1, 1:100; Developmental Studies Hybridoma Bank), K18 (1:200; gift from E. Fuchs Lab), p63a (H-129, 1:100; Santa Cruz Biotech, sc-8344), SMA (1:200; Sigma, A5228), Laminin (1:100; Thermo Scientific, RB-082-A1), K15 (1:100; Thermo Scientific MS-1068-P1), Na⁺/K⁺ ATPase (1:300; abcam ab58475), K5 (1:300; gift from C. Jamora), Ki67 1:200 (Leica, NCL-Ki67p), P-Smad1/5/8 1:50 (Cell Signaling, 9511), BrdU 1:200 (abcam ab6326), K1 1:300 (gift from C. Jamora), Loricrin 1:300 (gift from C. Jamora), AE15 1:100 (Santa Cruz Biotech, sc57012), AE13 1:100 (Santa Cruz Biotech, sc80607). Secondary antibodies: Rabbit anti-Rat TRITC 1:300 (Sigma T4280), Goat anti-Rabbit TRITC 1:300 (Sigma T6778), Goat anti-Mouse TRITC 1:300 (Sigma T6528), Alexa 594 Goat anti-Chicken 1:500 (Invitrogen A11042), Alexa 488 Goat anti-Chicken 1:500 (Invitrogen A11039), Goat anti-Rabbit FITC 1:300 (Sigma F9887), Alexa 350 Goat anti-Rat 1:150 (Invitrogen A21093), Alexa 350 Donkey anti-Rabbit 1:150 (Invitrogen A10039).

### Isolation of SG LRCs and Sole's Epidermal Basal Cells

KSTetOff/TreH2BGFP animals were fed 1 mg/g doxycycline food for 4 weeks starting around P21-28. GFP+ sweat glands were dissected out with its surrounding sole's epidermis from the fingertips of 20-30 mice and treated with 1000 U/ml Collagenase type I for 1 h at 37° C. with shaking Sweat glands were mechanically separated from its epidermis and treated further with 1000 U/ml Collagenase type I and 500 μg/μl Hyaluronidase (Sigma) for 1 h at 37° C. with shaking Purified sole's epidermis and SGs were independently washed with DPBS and digested in 0.25% Trypsin-EDTA for 20 min at 37° C. with shaking Neutralize and filter cells through a 40 um cell strainer.

### FACS

For FACS, isolated 4 weeks chased H2BGFP labeled sweat gland cells were stained with a primary antibody: anti-α6 integrin (CD49f) conjugated to PE (1:200; BD Pharmingen) for 30 min and sorted using the FACS Aria II cell sorter (BD, Bioscience) for H2BGFP+/α6+ and H2BGFP−/α6+ populations. Cells were collected in RNAprotect Cell Reagent (Qiagen) for later RNA isolation. Similarly, FACS analysis were performed on isolated K15-GFP labeled sweat gland cells, stained with the primary antibody against anti-α6 integrin as described above.

### Chamber Graft

A silicon chamber was implanted onto the backs of immunocompromised “nude” mice with a full-thickness skin wound as previously described (Weinberg, W. C. et al. (1993) J Invest Dermatol. 100:229-236). 4 weeks chased whole H2BGFP sweat glands were dissected out with its surrounding sole's epidermis from the fingertips and treated with 1000 U/ml Collagenase type I for 1 h at 37° C. with shaking After separation from the sole's epidermis, purified dermis with remaining sweat glands were transplanted into the humidified silicone chamber. The upper chamber was removed 2 weeks after transplantation and the bottom half is removed 3 weeks after transplantation as the skin is healing. The nude mice were either fed regular mouse diet or doxycycline food after transplantation for the duration of the experiments.

Similarly, dissociated unsorted 4 weeks chased SG single cell suspension labeled with H2BGFP after separation from the sole's epidermis was mixed with freshly isolated unmarked newborn dermal fibroblasts (approximately 6 million cells total) at a 1:1 proportion and injected into the chamber. Mice were sacrificed and samples from the graft regions were taken for GFP+ expression and tissue analysis. All mice work was conducted according to the Institutional Animal Care and Use Committee (IACUC) at the University of Southern California. The protocols (No. 11306 and 11325) were approved by the IACUC Committee. All surgery was performed under either isoflurane or ketamine anesthesia and all efforts were made to minimize suffering with analgesics (Buprenex prior and post-surgery was administrated).

### Subcutaneous Injections

Dissociated unsorted SG single cell suspension labeled with H2BGFP after 4 weeks of chase after separation from the sole's epidermis was mixed with freshly isolated unmarked newborn dermal fibroblasts at a 1:1 proportion and injected subcutaneously underneath the back skin of an immunocompromised “nude” mouse (approximately 1.5 million cells per spot).

Confocal Microscopy of sweat gland LRCs and 3D reconstruction of sweat glandsTissue was imaged on a Zeiss (Carl Zeiss, LLC) Axiovert 200 inverted microscope with an LSM 510 meta confocal scan head using a 40×/1.2NA water immersion lens. Tissue was dissected, stained and placed in a 35 mm glass bottom tissue culture dish (MatTek Corporation, Asland, Md.). Tissues were submerged in deionized water to minimize refractivity. Histone 2B GFP and TRITC stained laminin (1:100; Thermo Scientific, RB-082-A1) were visualized using conventional confocal imaging using argon laser lines at 488 nm and 543 nm, respectively. DAPI was imaged with 2-photon excitation using a Coherent Chameleon (Coherent Inc, Santa Clara, Calif.), pulsed laser tuned to 800 nm. Images were collected at 0.22 μm in plane (xy) and optically sectioned at 2 μm (in z). 3D reconstruction and visualizations were performed in ImageJ (http://rsbweb.nih.gov/ij/), Fiji (http://fiji.sc/wiki/index.php/Fiji), Avizo 6.3 (VSG, Burlinton, Mass.) and Vaa3D (http://www.vaa3d.org/).

RNA Isolation and qPCR

Total RNAs were purified from FACS-sorted SG LRCs, SG non-LRCs, and the basal layer of the sole's epidermis using Qiagen's RNeasy Micro kit according to the manufacturer's instructions. Equal amounts of RNA were reverse transcribed using the Superscript III First-Strand Synthesis System (Invitrogen) according to the manufacturer's instructions. cDNAs were amplified by PCR and used in triplicate for each qPCR sample primer set with all primer sets designed to work under the same conditions. Real-time PCR amplification of particular genes of interest was performed using an Applied Biosystems 7900HT Fast Real-Time PCR System and the fold difference between samples and controls were calculated based on the 2^{-ΔΔCT} method, normalized to β-actin levels.

### Microarray Analysis

Total RNAs from FACS of SG LRCs (GFP+/α6 integrin+), SG non-LRCs (GFP−/α6 integrin+), and sole's epidermis (α6 integrin+) were purified using a RNeasy Micro Kit (Qiagen, Valencia Calif., United States), and quantified (Nanodrop, United States) for two separate microarray analysis from two independent biological samples. RNA 6000 Pico Assay (Agilent Technologies, Palo Alto, Calif., United States) was used for RNA quality check. Amplification/labeling were performed on 50 nanogram (ng) and 250 ng to obtain biotinylated cRNA (Ovation™ RNA Amplification System; Nugen, San Carlos, Calif., United States and Ambion Kit; Affymetrix, Santa Clara, Calif., United States, respectively), and either 3.75 μg or 5.5 μg ssDNA were used for fragmentation, labeling and hybridization. Hybridization was performed at 45° C. for 18 h to Mouse Gene 1.0 ST array (Affymetrix, Santa Clara, Calif., United States). Processed chips were read by GeneChip Scanner 3000 7G (Genomics Core Facility, Children's Hospital Los Angeles, Los Angeles, Calif., United States). The raw expression intensity data was imported into Partek Genomic Suite v6 (Partek Inc., St. Louis, Mo., United States). The data was pre-processed using the RMA algorithm with the default Partek setting. Following fold change calculations, differentially expressed gene (DEG) lists containing probe sets with 2-fold intensity changes in either direction were generated. Common DEG list was generated by comparing the DEG list of the sweat gland LRCs experiment and GFP-α6+ sweat glands experiment to the sole's epidermis. Functional annotation of the DEG list was carried out using the “Database for Annotation, Visualization and Integrated Discovery” (DAVID). The microarray data are available in a public GEO database with an accession number (#GSE49011).

### Results

Slow Cycling LRCs are Localized to the Proximal—Acinar Region of Sweat Glands.

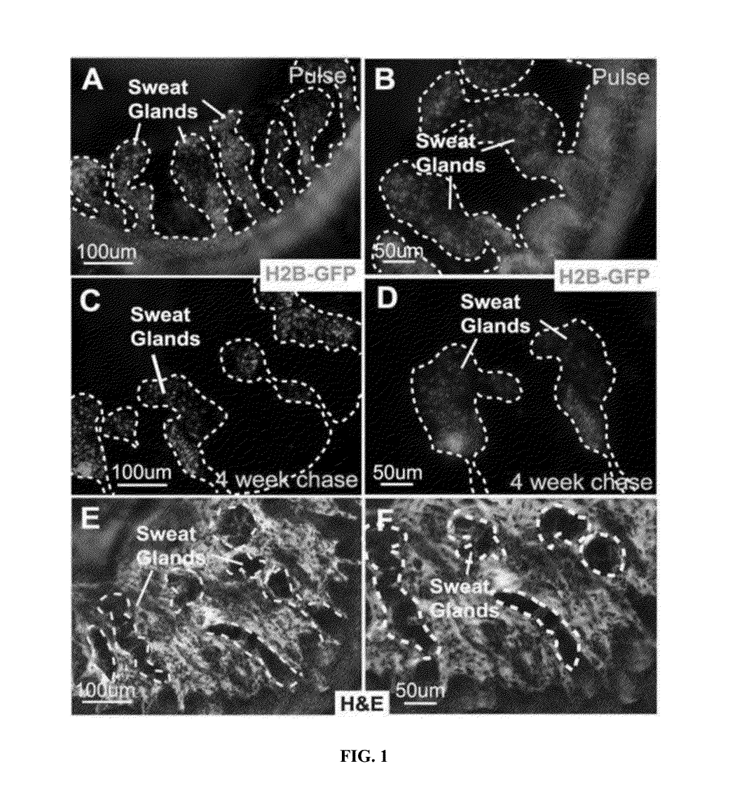

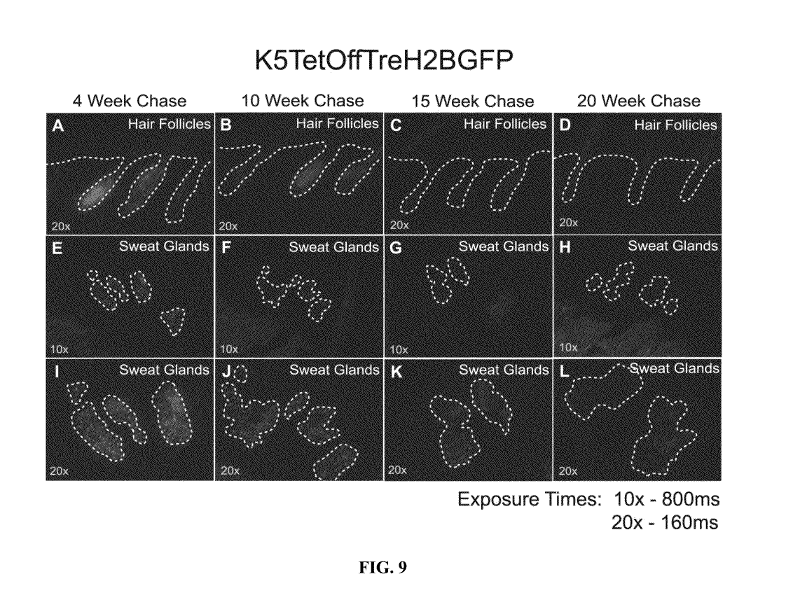

Applicant employed the recently developed H2BGFP system, composed of two transgenic mouse lines: keratin 5-driven tetracycline repressor mice (K5-tTA) (Diamond, I. et al. (2000) J Invest Dermatol. 115:788-794) and tetracycline response element-driven histone H2B-GFP transgenic mice (pTRE-H2B-GFP) (Tumbar, T. et al. (2004) Science 303:359-363), to detect live, slow cycling LRCs in vivo. In these animals, H2BGFP expression was uniformly detected in all cells of the epidermis, hf and SGs (ducts and glands) prior to Doxy treatment (FIG. 1, Panels A and B). After 4 weeks of “chase” experiments by switching off H2BGFP expression with Doxy treatment beginning at 3 to 4 weeks of age, the presence of infrequently dividing LRCs in SGs (FIG. 1, Panels C and D) was demonstrated. These SG LRCs are extremely slow cycling with some of them persisting for more than 20 weeks of chase (FIG. 9). The H2BGFP expression of LRCs was restricted to the proximal acinar gland region with no expression in the ductal region (distal part of the SGs connected to the overlying epidermis) (FIG. 1, Panels C and D). This region was previously reported to contain LRCs in human eccrine glands (Nakamura, M. et al. (2009) J Invest Dermatol. 129:2077-2078). As an internal control, Applicant observed H2BGFP expression of LRCs restricted to hfSCs (bulge) after Doxy treatment (data not shown). Histological localization of LRCs in SGs was confirmed on serial sections of the paw region by hematoxylin and eosin staining (H&E) (FIG. 1, Panels E and F).

Sweat Gland LRCs are Attached to the Basement Membrane and Demonstrate Myoepithelial Characteristics.

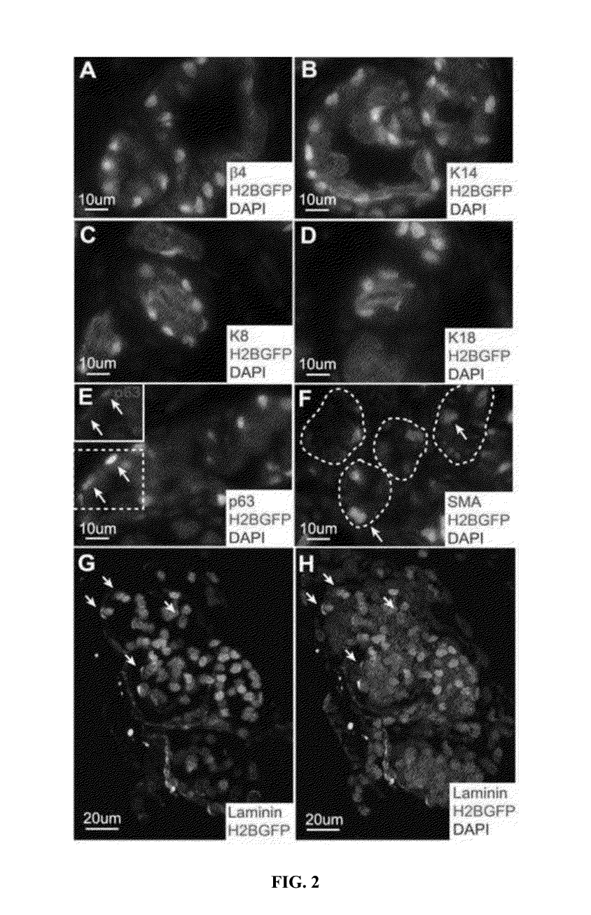

SGs are composed of three different cell types, dark apical cells of the lumen, clear and myoepithelial cells of the basal layer. Therefore, Applicant used immunofluorescence staining with a number of different markers to determine where SG LRCs are localized. Applicant demonstrated that these SG LRCs are attached to the basement membrane expressing β4 integrin (FIG. 2, Panel A). In addition, these SG LRCs also co-expressed the basal layer marker keratin 14 (K14) (FIG. 2, Panel B), whereas lumenal layer markers, keratin 8 (K8) and keratin 18 (K18), did not overlap with SG LRCs (FIG. 2, Panels C and D, respectively). Next, by performing p63 antibody (Ab) staining for a mammary gland myoepithelial cell marker (Barbareschi, M. et al. (2001) Am J Surg Pathol. 25:1054-1060), which has also been shown to be important for epidermal self-renewal and differentiation (Koster, M. I. et al. (2004) Genes Dev 18:126-131), Applicant distinguished that SG LRCs specifically co-localize with myoepithelial cells of the basal layer (FIG. 2, Panel E). In addition, co-localization of smooth muscle actin (SMA) with SG LRCs was shown, which further support that SG LRCs have a myoepithelial cell characteristic with basal layer localization (FIG. 2, Panel F).

SGs exist as 3-dimensional (3D) structures therefore, Applicant performed whole mount staining of 4 weeks chased SGs with a basement membrane marker, laminin, (with DAPI nuclear counterstain) to examine how these LRCs are organized within this appendage (FIG. 2, Panels G and H). A two-photon confocal microscopy was used to acquire serial Z-stacks and performed 3D re-construction of enzymatically purified whole SGs (see Material and Method for details). This allowed Applicant to generate a 3D model of SGs to visualize the 3D organization of LRCs attached to the basement membrane organized in a scattered fashion (FIG. 2, Panels G and H, arrows). This contrasts with the clustered LRCs organization found in the hair follicle and cornea limbus.

Sweat Gland LRCs Express Keratin 15 (K15) in the Acinar Region and K15 Marked Cells Contribute Long Term to the Sweat Gland Structure but not to Epidermal Homeostasis.

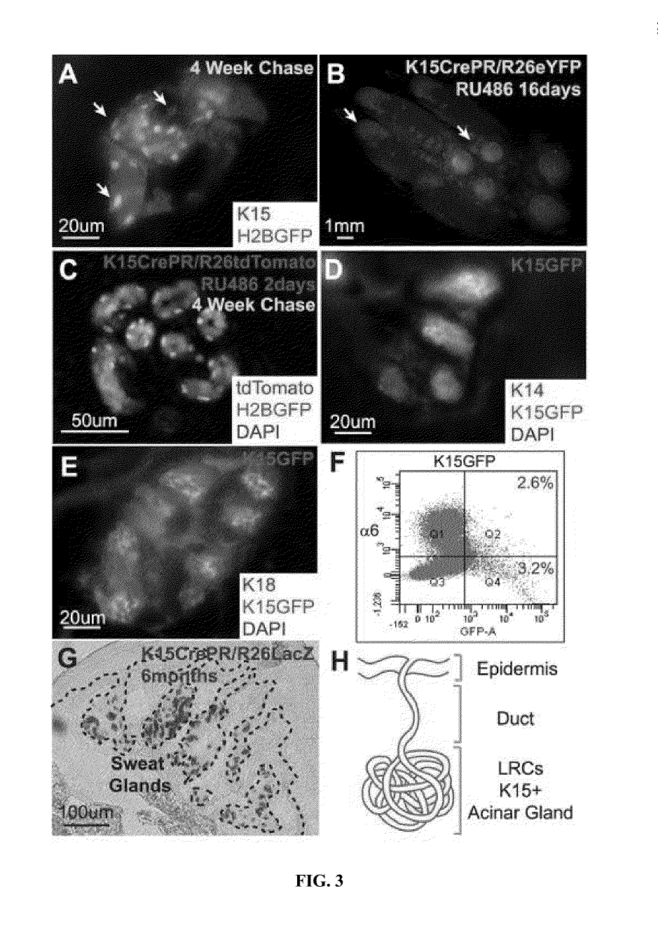

It has been previously shown that another LRC population located in the hf bulge specifically expresses K15 (Morris, R. J. et al. (2004) Nat Biotechnol. 22:411-417), therefore, Applicant examined whether K15 expression is also present in SGs. First, using K15 Ab staining, Applicant demonstrated that mouse SG LRCs co-localized with K15 (FIG. 3, Panel A) confirming previously published data for human eccrine gland LRCs (Nakamura, M. et al. (2009) J Invest Dermatol. 129:2077-2078). Next, Applicant established a genetic lineage tracing approach to monitor K15 expressing cells in SGs by generating a K15 driven Cre recombinase conjugated with a truncated progesterone receptor (PR), (K15CrePR) (Morris, R. J. et al. (2004) Nat Biotechnol. 22:411-417), with three different Cre-dependent Rosa26 reporter mice: eYFP (enhanced Yellow Fluorescent Protein, R26eYFP) (Srinivas, S. et al. (2001) BMC Dev Biol. 1:4), tdTomato (tandem dimer Tomato, R26tdTom) (Madisen, L. et al. (2010) Nat Neurosci. 13:133-140) and LacZ (β-galactosidase, R26LacZ) (Soriano, P. (1999) Nat Genet. 21:70-71). After RU486 (RU) treatment for 16 days beginning at P43, YFP expression was detected in SGs of the palm in both the foot pads and fingertips (FIG. 3, Panel B, arrows). This system allowed Applicant to mark initially K15 positive cells and permanently label all of its progeny in SG structures.

To demonstrate whether K15 marked cells in SGs co-localized with LRCs, Applicant used a K15CrePR system with a Rosa26-tdTomato reporter mouse crossed onto the KSTetOff/TreH2BGFP background. After 4 weeks of chase with Doxy treatment (at ˜P49) when LRCs (green) were present in SGs, Applicant labeled the K15 expressing cells using a short RU treatment for 2 days to mark K15 positive cells in SGs with tdTomato expression (FIG. 3, Panel C). These results revealed that tdTomato expression (red) was restricted to the acinar regions of the SGs and co-localized with LRCs in the basal layer (FIG. 3, Panel C). Applicant also used a K15-GFP (Green Fluorescent Protein) reporter system (Morris, R. J. et al. (2004) Nat Biotechnol. 22:411-417) to visualize cells which actively expressed K15 (without permanently labeling their progenies) and confirmed that K15-GFP expressing cells were localized in the acinar region of SGs (FIG. 3, Panels D and E) where LRCs are found (FIG. 3, Panels A and C). Although K15 expression includes the LRCs population, they are not specific to SG LRCs. Applicant stained K15-GFP SGs for the basal layer marker, K14, and the lumenal layer marker, K18, to demonstrate that K15 positive SG cells marked both basal and lumenal layers of the SG structure in adult mice (FIG. 3, Panels D and E). Additionally, fluorescence activated cell sorting (FACS) analysis from K15-GFP mice demonstrated that less than 6% of the total number of isolated cells from SG regions were GFP positive (FIG. 3, Panel F). Approximately less than half of the K15-GFP positive cells (2.6% out of 5.8%) were labeled by α6-integrin, a basal layer marker, with the remaining half (3.2% out of 5.8%) of K15-GFP positive SG cells negative for α6-integrin (FIG. 3, Panel F), which are presumably K15 positive lumenal cells. Previously, Lu et al. have used this same K15CrePR promoter to identify progenitor cells of the lumenal layer (Lu, C. P. et al. (2012) Cell 150:136-150). However, K15 expression was activated at an early postnatal stage (P5) prior the completion of SG morphogenesis. Here, Applicant analyzed K15 expression in mature SGs of adult mice and showed that K15 targets the lumenal layer of SGs consistent with the previous report. However, by inducing K15 expression in adult mice, Applicant shows that K15 can also target basal layer cells in addition to lumenal cells. Finally, to address the long term contribution of K15 expressing cells in SG and epidermal homeostasis, Applicant used K15CrePR mice crossed with a R26LacZ reporter. After induction of LacZ by 16 days of RU treatment in adult mice, Applicant was able to detect LacZ positive cells (with X-gal staining) from the time of RU treatment to more than 6 months after initial transgene activation in the acinar region (FIG. 3, Panel G). This demonstrated that K15 positive SG cells could survive long term and contribute to SG homeostasis. However, LacZ positive cells were not present in the surrounding epidermis or distal SG duct (FIG. 3, Panel G) suggesting that these cells do not contribute towards physiological epidermal homeostasis behaving similar to hfs (Ito, M. et al. (2005) Nat Med. 11:1351-1354).

Isolating LRCs from Sweat Glands

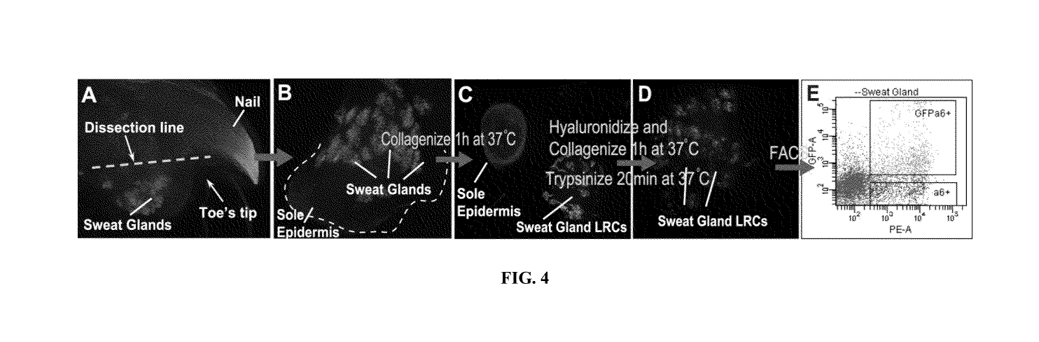

To isolate pure fractions of SG LRCs, Applicant used a combination of surgical dissection with subsequent enzymatic digestions. To avoid contamination from hf LRCs, Applicant collected the whole paw with the toes and dissected out SGs with the surrounding sole's epidermis (FIG. 4, Panel A, yellow dissection line and FIG. 4, Panel B). To purify SGs from the attached sole's epidermis, Applicant then digested the dissected SGs with collagenase for 1 hour at 37° C. and mechanically separated the epidermis (FIG. 4, Panel C). Purified SGs were further digested with collagenase and hyaluronidase at 37° C. for 1 hour and then trypsinized for 20 min at 37° C. to obtain a single cell suspension ready for FACS (FIG. 4, Panel D). Since SG LRCs were attached to the basement membrane, Applicant stained these cells with FACS specific antibody against α6 integrin to help purify these live LRCs and adjacent non-LRC basal cells. The specific FACS gates to sort double positive SG LRCs (H2BGFP+ and α6 integrin+) and single positive SG non-LRCs (H2BGFP- and α6 integrin+) were setup according to negative (unstained), single GFP positive and single a 6 integrin positive control cells (FIG. 4, Panel E). The majority of SG LRCs (H2BGFP+) were positive for α6 integrin (FIG. 4, Panel E). In parallel, Applicant isolated adjacent SG non-LRC basal cells (α6 integrin+) (FIG. 4, Panel E). FACS analysis revealed that SG LRCs accounted for approximately 1-6% of the whole dermal fraction containing the SG skin appendage (after detachment of the sole's epidermis).

### Defining the Sweat Gland LRCs Characteristic

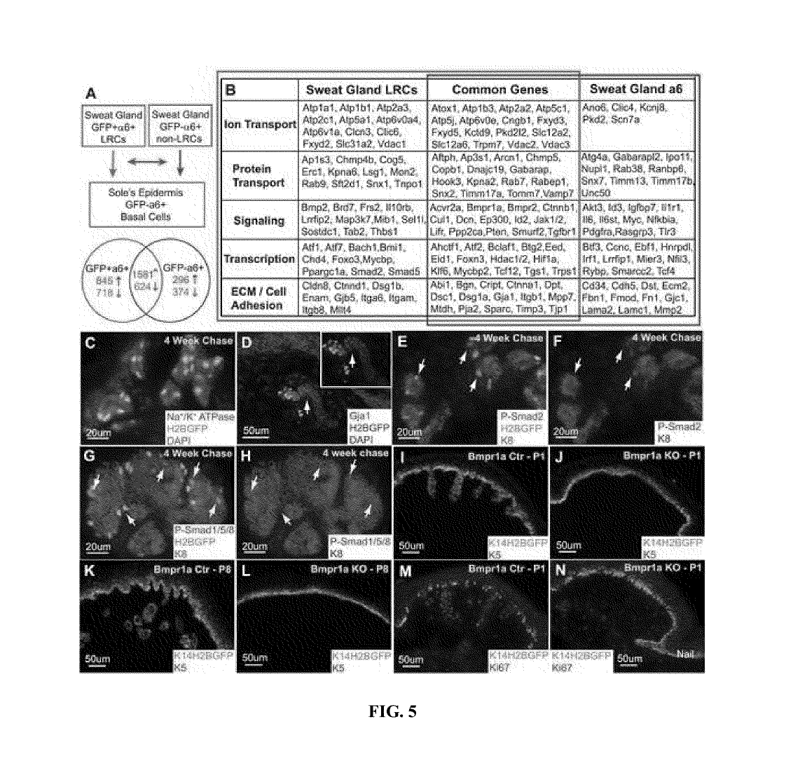

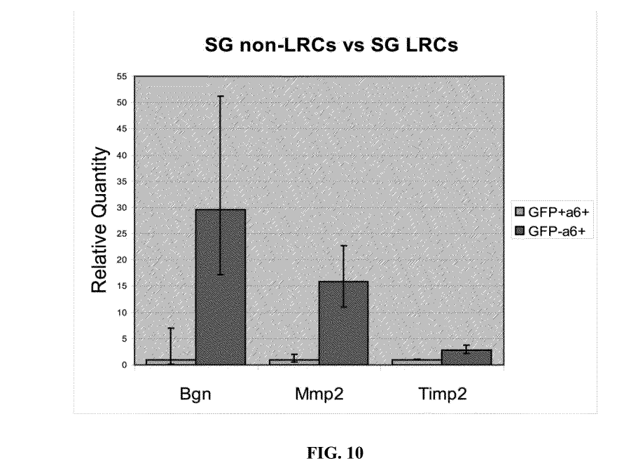

To identify the transcriptional gene expression profile of SG basal layer cells, total RNA was extracted from the GFP+/α6+ population (SG LRCs), GFP−/α6+ adjacent basal layer cells (SG non-LRCs), and α6+ basal layer cells of the sole's epidermis for microarray analyses from two independent experiments. Purification and microarray hybridization (Affymetrix Mouse Gene 1.0ST) of each fraction were performed in duplicate. Then, each population, SG LRCs and non-LRCs, were compared separately to the basal layer of the sole's epidermis (FIG. 5, Panel A). By comparing SG LRCs to sole's epidermal basal layer (α6+), Applicant identified 2426 (845+1581) genes to be consistently up-regulated and 1342 (718+624) genes to be down-regulated by at least 2 fold in two independent microarray analyses. Subsequently, Applicant compared non-LRCs to the sole's epidermal basal layer (α6+) and identified 1877 (296+1581) genes which were consistently up-regulated and 998 (374+624) genes down-regulated by at least 2-fold (FIG. 5, Panel A). Applicant then examined how many of the gene changes identified in SG LRCs and SG non-LRCs were commonly or uniquely expressed between both populations. Applicant identified 1581 up- and 624 down-regulated genes (out of 2,205 total) to be commonly up-regulated or down-regulated by at least 2-fold in two independent microarray analyses from both GFP+/α6+ and GFP−/α6+ basal layer cells of the SG (FIG. 5, Panel A and Table 1). Moreover, 1563 genes (845 up- and 718 down-regulated) were uniquely expressed in SG LRCs (FIG. 5, Panel A and Table 2), whereas 670 (296 up and 374 down-regulated) genes were uniquely expressed in SG non-LRCs (FIG. 5, Panel A and Table 3). All these genes were consistently regulated in the two independent experiments conducted. Some of the genes were validated through real-time PCR using separately isolated biological samples from 2-3 independent experiments independent from the microarray analyses. In particular, genes expressed in the SG GFP-α6+ non-LRCs, Mmp2 and Timp2, were confirmed to be up-regulated through real-time PCR when compared to the SG LRCs fraction (FIG. 10). In addition, Applicant also performed real-time PCR for biglycan which was found to be up-regulated in both SG LRCs and non-LRCs in the microarray analyses. Although biglycan was found to be commonly expressed in both fractions, it was up-regulated by at least 2-fold more in the non-LRCs fraction compared to the LRCs population. Accordingly, this real-time PCR data validates that biglycan is up-regulated in SG GFP-α6+ non-LRCs when compared to SG LRCs. All real-time data was performed in triplicate using cDNA from either 2 or 3 independent biological samples.

As SG LRCs showed myoepithelial characteristics, Applicant further probed how the gene expression profile in this population corresponded to its function when compared to SG non-LRCs basal cells. To this end, Applicant performed functional annotations (grouped according to the DAVID software) which enabled us to categorize a number of identified genes in LRCs and non-LRCs (FIG. 5, Panel B). Applicant found that a number of these genes were involved in cell adhesion, signaling and transcription. Moreover, a number of transporters and ion channels, important for SG function, were identified in the microarray analyses (FIG. 5, Panel B). In particular, Applicant identified the expression of sodium/potassium (Na⁺/K⁺) ATPase pumps (Atp1a1, Atp1b1, Atp1b3) in both GFP+/α6+ and GFP−/α6+ cells of the SGs. This expression was confirmed by Na⁺/K⁺ ATPase Ab staining in the SG skin appendage after a 4 weeks chase with Doxy (FIG. 5, Panel C). In addition, vimentin was found to be commonly up-regulated in LRCs and non-LRCs, which was previously reported in the human SG myoepithelium (Schon, M. et al. (1999) J Cell Sci. 112(Pt 12):1925-1936). To further validate the genes identified in the microarray, Applicant stained 4 weeks chased SGs with gap junction protein, alpha 1 (Gja1), which was up-regulated in both SG LRCs and non-LRCs, and confirmed its co-localization with LRCs (FIG. 5, Panel D, arrow) and non-LRCs of the sweat duct. Positive phopho-Smad2 (p-Smad2) expression in SG LRCs and lumenal cells also confirms its up-regulation in the microarray (FIG. 5, Panels E and F). Furthermore, a number of genes important for Bone Morphogenetic Protein (BMP) signaling, including Bmpr1a, Bmpr2, Smad5, Id2, Id3 and Decorin were up-regulated in the SG when compared to the sole's epidermis (FIG. 5, Panel B). Therefore, Applicant tested for BMP signaling activity using phospho-smad1/5/8 staining and observed positive nuclear phospho-Smad activity in adult mouse SGs suggesting that BMP signaling may be important in this appendage. More specifically, Applicant show nuclear phospho-smad1/5/8 expression in some LRCs (FIG. 5, Panels G and H, arrows). However, its expression was not specific to LRCs and was also found in other SG cells including lumenal cells. To functionally examine the effects of BMP signaling, Applicant ablated Bmpr1a during development using a keratin 14 driven Cre recombinase (K14Cre) (Vasioukhin, V. et al. (1999) Proc Natl Acad Sci. USA 96:8551-8556) crossed onto a K14-H2BGFP reporter (Rendl, M. et al. (2005) PLoS Biol 3:e331). At P1 and P8, Applicant observed downgrowth and SG development in control mice (FIG. 5, Panels E and G). However, in Bmpr1a KO mice, SGs were absent and failed to form suggesting that BMP signaling is required for normal SG morphogenesis (FIG. 5, Panels F and H). Ki67 staining indicated that the basal layer of Bmpr1a KO epidermis appeared capable of proliferation, but failed to acquire the SG fate (FIG. 5, Panels I and J).

Acinar Sweat Gland Cells do not Contribute to the Epidermis During Wound Healing.

Previously, it has been reported that bulge hfSCs with LRCs characteristic do not participate in epidermal homeostasis, however, they can actively deliver cells to the epidermal wound during skin injury (Ito, M. et al. (2005) Nat Med. 11:1351-1354). In addition, keratinocytes in the region directly above bulge LRCs, marked by Lgr6, can postnatally generate the sebaceous gland and interfollicular epidermis contributing to epidermal homeostasis and can execute long term wound repair (Snippert, H. J. et al. (2010) Science 327:1385-1389; Petersson, M. et al. (2011) EMBO J 30:3004-3018). However, little is known about the role of SGs in active epidermal regeneration initiated upon wounding. In human SGs, it has been reported that basal cells of the straight duct undergo division when provoked by skin injury (Lobitz, W. C., Jr. et al. (1954) J Invest Dermatol. 22:189-198).

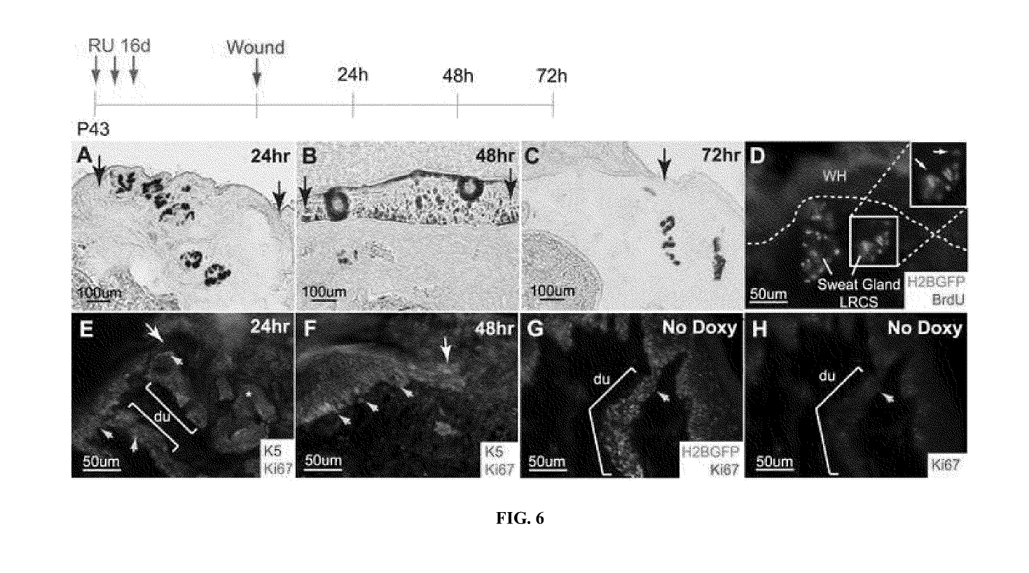

Since Applicant has demonstrated that SG acinar cells marked by K15CrePR/R26LacZ do not participate in epidermal keratinocyte lineages during homeostasis, Applicant next examined if these acinar SG cells could respond and actively contribute to epidermal wound repair upon injury. Wounds where the epidermis was effectively scraped off were performed on K15CrePR/R26LacZ mice in order to trace K15 positive SG cells and their progeny. Wounds were allowed to heal for 24 h, 48 h, and 72 h when samples were collected for analysis. X-gal staining for LacZ enabled visualization of K15 positive SG cells and their progeny (blue). At all time points, no blue cells were detected in the regenerating epidermis (FIG. 6, Panels A-C). To investigate whether SG LRCs were responding to the injury, Applicant performed similar wound healing experiments on 4 weeks chased KSTetOff/TreH2BGFP mice. In this experiment, Applicant pulsed the animals with BrdU to probe for proliferating cells in SGs. BrdU was detected sporadically in a few LRCs, but most SG cells remained quiescent (FIG. 6, Panel D). To examine which cells were responding to the injury, Applicant stained the wound healing samples from K15CrePR/R26LacZ mice with a Ki67 Ab and found that the basal layer of the epidermis and SG ductal cells were active in the cell cycle, whereas the SG acinar region remained quiescent (FIG. 6, Panels E and F). Under physiological homeostasis, SG duct cells appear to be more active than the SG acinar region similar to the basal layer of the epidermis (FIG. 6, Panels G and H).

Sweat Gland LRCs can Trans-Differentiate into the Epidermis Under Prolonged Isolated Wound Healing

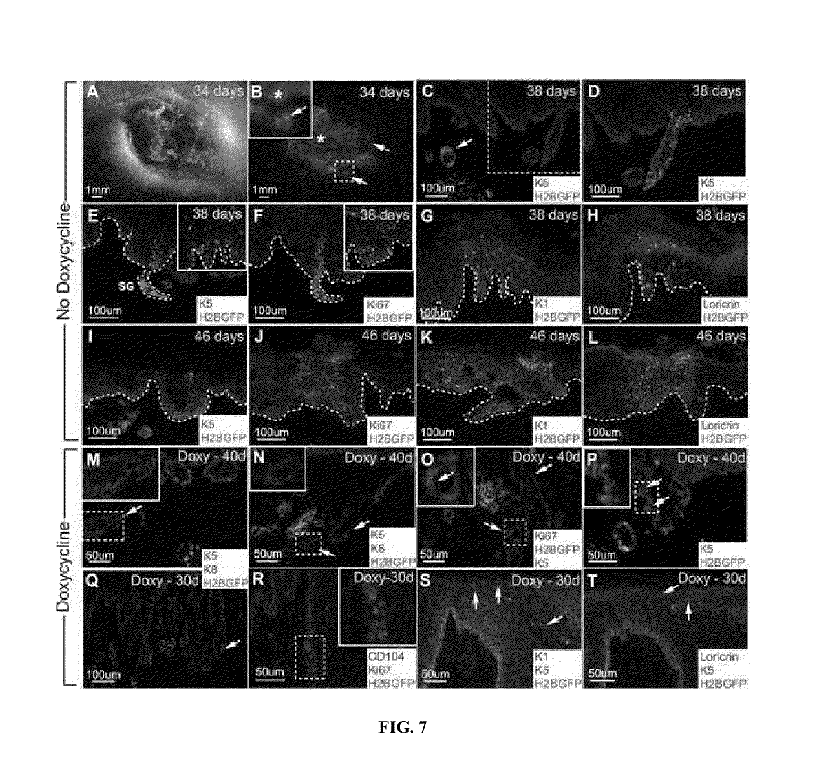

Although the SG acinar cells did not contribute to wound healing under normal circumstances, Applicant further challenged the system using more favorable conditions. For this, Applicant isolated 4 weeks chased H2BGFP labeled whole SGs through collagenase digestion (as described in FIG. 4, Panel B and FIG. 3, Panel C and as described herein). Then, Applicant transplanted the dermal portion of these SGs into a silicon chamber implanted onto the backs of immunocompromised “nude” mice (Weinberg, W. C. et al. (1993) J Invest Dermatol. 100:229-236). The advantage of this chamber graft experiment was that the transplanted dome containing the SGs physically separated and initially prevented the surrounding “nude” epidermis from closing the wound, thereby increasing the chance for the transplanted SG epithelial cells to respond to wound regeneration. The chamber was removed approximately 14 days after transplantation and the wound was allowed to heal for about one month (FIG. 7, Panel A). At 34 days after transplantation, H2BGFP labeled cells were observed in the transplanted region of the regenerated skin (FIG. 7, Panel B). To determine the participation of these marked SG cells in epidermal regeneration, Applicant sectioned and stained these transplants with a number of different markers at 38 and 46 days after transplantation. The majority of the transplanted H2BGFP marked SGs were found in the dermis (FIG. 7, Panel C, arrows). However, when Applicant closely examined the epidermis and increased exposure time for the GFP channel on the same section, Applicant found that a number of H2BGFP expressing cells with lower intensity contributed to the newly formed epidermis (FIG. 7, Panel C vs. D). These H2BGFP cells co-localized with the basal layer marker, keratin 5 (K5), at 38 and 46 days (FIG. 7, Panels D, E and I, respectively). Furthermore, Applicant demonstrated that these H2BGFP labeled SG cells could proliferate along the basal layer using Ki67 co-staining (FIG. 7, Panels F and J). Next, Applicant checked if the H2BGFP marked SG cells found in the regenerating epidermis were able to adopt epithelial characteristic by performing immunofluorescence staining for different epidermal differentiation markers. At both 38 days and 46 days, Applicant showed that the H2BGFP positive SG cells can contribute to the suprabasal layer of the epidermis indicated by co-localization with K1, a suprabasal layer marker (FIG. 7, Panels G and K). Similarly, Applicant observed that H2BGFP labeled cells could differentiate into cells of the granular layer indicated by loricrin staining (FIG. 7, Panels H and L). Thus, these lineage tracing experiments demonstrated that H2BGFP marked SG cells can contribute to epidermal regeneration following injury.

However, since these experiments were performed “off Doxy” Applicant was not able to rule out whether the SG H2BGFP LRCs themselves proliferated and contributed to this newly formed epidermis or whether other non-LRC SG cells “turned on” H2BGFP expression in the absence of Doxy. To address this, Applicant repeated this experiment using 4 weeks chased H2BGFP labeled SGs. In this case, the host mouse with transplanted SG dermis was kept on Doxy treatment for the entire experiment; thus, only SG LRCs and their direct descendents would be marked by H2BGFP. At 30 and 40 days after transplantation, the H2BGFP label appeared to have been diluted out of some acinar SG structures (confirmed by K8 lumenal layer staining) but not all (FIG. 7, Panels M, N, Q, arrows). Moreover, some of these transplanted SGs appeared to be fused with the newly regenerated epidermis (FIG. 7, Panels M and Q). In some instances, SGs closer to the SG-epidermal connections were the ones lacking H2BGFP marked SG LRCs, suggesting that the LRCs of these SGs had been actively dividing and diluted out the nuclear H2BGFP label (FIG. 7, Panels N and Q, arrows). This observation was confirmed using Ki67 staining where Applicant observed Ki67 positive cells present in SGs lacking LRCs as marked by green K5 membrane staining (FIG. 7, Panel 0). Finally, Applicant shows that these SG LRCs themselves can also contribute to the regeneration of the epidermis as marked with K5 co-localization (FIG. 7, Panel P, arrows). Staining for Ki67, Applicant illustrates that SG LRCs near the basal layer of the newly regenerated epidermis, marked by basement membrane marker β4 integrin (CD104), can proliferate (FIG. 7, Panel R). In addition, SG LRCs can differentiate into cells of the suprabasal and granular layers of the epidermis as marked by K1 and loricrin co-localization, respectively (FIG. 7, Panels S and T). In conclusion, Applicant has shown that under more favorable conditions, SG LRCs can divide and contribute to the different layers of the epidermis. In total, Applicant performed five chamber graft experiments with whole SGs where three were successful, consistently demonstrating the incorporation of H2BGFP marked SG cells into the epidermis.

### Dissociated Sweat Gland Cells can Regenerate Both Sweat Gland and Hair Follicle Appendages

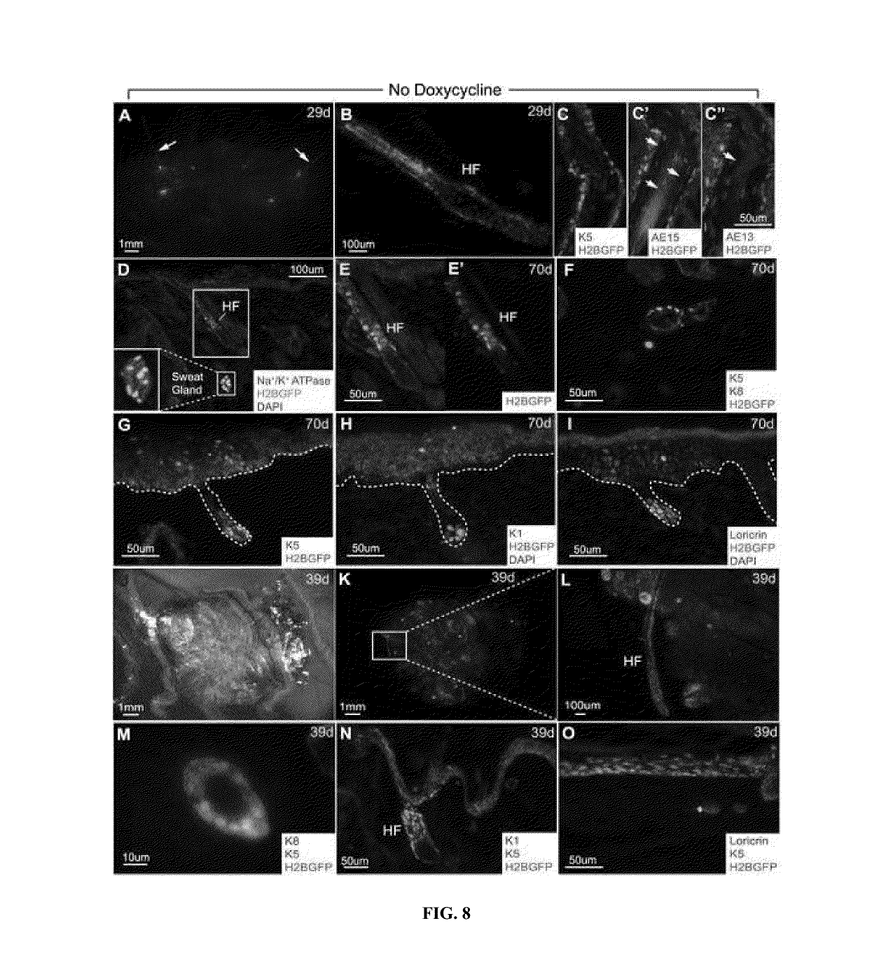

Finally, since Applicant was unable to passage and expand these KSTetOff/TreH2BGFP cells in culture, Applicant could not probe their in vitro potential and subsequently use them for in vivo reconstitution assays. Instead, Applicant used unsorted dissociated SG cells isolated directly from whole SGs. To further probe the regenerative potential of all SG cells, Applicant dissociated 4 weeks chased, H2BGFP labeled, SGs into a single cell suspension after separation from the sole's epidermis (as in FIG. 4, Panels B and C). Next, Applicant performed chamber graft transplantation by mixing these unsorted H2BGFP marked dissociated SG cells with unmarked freshly isolated back skin dermal fibroblasts. Surprisingly, 29 days after transplantation, Applicant observed several GFP positive areas under the epidermis with some of them connected to GFP positive hair-like fibers sticking out of the graft region (FIG. 8, Panel A). Indeed, analysis of sections from the graft area confirmed the presence of GFP positive hfs, likely originating from the transplanted unsorted H2BGFP labeled SG single cells suspension (FIG. 8, Panel B). These newly formed hfs were further characterized by immunofluorescence staining with several hair specific markers including K5 positive expression in the outer root sheath (FIG. 8, Panel C), AE15 expression in the inner root sheath and medulla (FIG. 8, Panel C′), and AE13 expression in the cortex of the hair shaft (FIG. 8, Panel C″). Interestingly, when Applicant analyzed the graft 70 days after transplantation, Applicant also found coexisting (on the same section) H2BGFP positive SG structures expressing SG markers: Na⁺/K⁺ ATPase and lumenal layer marker K8 (FIG. 8, Panels D, inset and F, respectively) in addition to GFP marked hfs (FIG. 8, Panels D-E′). This demonstrated that dissociated single SG cells suspension can still be flexible in their fate decision choices between SGs or hfs. Both these structures were found to coexist in the same region which physiologically is not observed in mice. Consistent with the rest of the SG graft data presented here, the single SG cells suspension can also contribute to the regeneration of differentiated layers of the epidermis marked by K5, K1, and loricrin (FIG. 8, Panels G-I). In an independent experiment, Applicant also subcutaneously injected unsorted dissociated cells from H2BGFP labeled SGs (chased for 4 weeks) after separation from the sole's epidermis in combination with freshly isolated dermal fibroblasts under the back skin of an immunocompromised “nude” mouse. This transplant was harvested 39 days after subcutaneous injection where Applicant observed a cluster of hfs and GFP positive structures at the injection sites (FIG. 8, Panels J-K). Similar to the chamber graft experiment with dermal fibroblasts, Applicant observed the presence of GFP positive hfs differentiated from the injected H2BGFP positive SG cells (FIG. 8, Panel L). In addition, Applicant also found H2BGFP labeled SG structures containing both basal and lumenal layers marked by K5 and K8, respectively (FIG. 8, Panel M). These injected cells have again differentiated into the epidermis marked by K5, K1, and loricrin (FIG. 8, Panels N and O). Contamination from hfSCs are unlikely since hfs are absent in the palms where SGs are carefully dissected out. As a control, unsorted epidermal cells simultaneously isolated from the sole's epidermis were also subcutaneously injected with freshly isolated dermal fibroblasts; however, no signs of H2BGFP marked hf formation were observed (data not shown) suggesting that these newly formed hf and SGs are derived from SG cells.

### DISCUSSION

Here, Applicant demonstrates that cells with slow cycling characteristic exist in SGs as a scattered population localized in the SG basal layer of the proximal acinar region. As hair follicle LRCs have been previously described as SCs (Tumbar, T. et al. (2004) Science 303:359-363), Applicant asked if these newly identified SG LRCs also possess bona fide stem cell characteristics in vivo. Although LRCs have been reported in both mouse and human SGs (Nakamura, M. et al. (2009) J Invest Dermatol. 129:2077-2078), their characterization, precise localization and function has not been addressed so far. The KSTetOff/TreH2BGFP approach allows us to mark and isolate live SG LRCs in vivo for further characterization. Thus, Applicant was able to localize LRCs in the basal layer of the proximal acinar part of SGs and demonstrate their myoepithelial characteristic by SMA co-expression. In addition, Applicant demonstrated that SG LRCs specifically co-localize with p63 expression which has been shown to be specifically expressed in mammary gland myoepithelial cells (Barbareschi, M. et al. (2001) Am J Surg Pathol. 25:1054-1060). Previous studies illustrated that p63 is not only essential for epithelial development, but is also important for epidermal self-renewal and differentiation (Koster, M. I. et al. (2004) Genes Dev 18:126-131; Yang, A. et al. (1999) Nature 398:714-718). In addition, p63 is believed to be a marker of corneal and epidermal SCs (Pellegrini, G. et al. (2001) Proc Natl Acad Sci. USA 98:3156-3161).

Recently, some similar findings regarding SG LRCs were reported by Lu et al. (Lu, C. P. et al. (2012) Cell 150:136-150). However, they did not further characterize this SG LRC population or address their in vivo function. Instead, they employed elegant systems, previously published in mammary glands (Van Keymeulen, A. et al. (2011) Nature 479:189-193), to identify and characterize distinct SC populations in the basal and lumenal layers of SGs (Lu, C. P. et al. (2012) Cell 150:136-150). In Applicant's study, Applicant used a different approach and focused predominantly on basal, myoepithelial LRCs (GFP+/α6+) after 4 weeks of chase. Although Applicant was able to characterize this population of SG LRCs, this genetic approach did not allow Applicant to study the lumenal layer of SGs in more detail.

### The Proximal Acinar SG Region Only Contributes to its Own Structure During Homeostasis and Wound Healing

As an alternative and parallel approach, Applicant used genetic K15CrePR in vivo systems to mark cells specifically in the proximal acinar part of SGs including SG LRCs in the basal layer (FIG. 3, Panel C). However, these K15 labeled cells did not specifically mark SG LRCs, but had also marked cells of the lumenal layer in the proximal acinar part of SGs as illustrated with K15-GFP reporter mice (FIG. 3, Panels D and E). This data was confirmed by FACS analysis of SGs from adult K15-GFP reporter mice where Applicant observed an approximate 1:1 proportion of GFP marked basal and lumenal cells (FIG. 3, Panel F). Although Applicant's finding differs from data recently published by Lu et al., this discrepancy could be attributed to the use of different time points for labeling these K15 progenitors. In Applicant's case, Applicant marked them by RU treatment for K15CrePR systems or analyzed them in K15-GFP reporter mice during adulthood at/or after P21 (after SG morphogenesis was completed), whereas Lu et al. labeled them at an early postnatal time point between P5 to P9 (during SGs morphogenesis) and analyzed them at P22 (Lu, C. P. et al. (2012) Cell 150:136-150). Thus, it appears that this K15 promoter has different specificity during and after SG morphogenesis.

Since the K15CrePR system permanently marks K15 expressing cells and its progeny, Applicant used it to evaluate the contribution of K15 marked acinar cells in overall SG and skin homeostasis. Applicant's results demonstrate that K15 labeled cells localized exclusively in the acinar part of SGs and contributed long term to only the proximal glandular part, but not to homeostasis of the distal SG ducts or the surrounding epidermis (FIG. 3, Panel G). Furthermore, Applicant also shows that typical wound stimulation surprisingly did not activate these proximal acinar SG cells to participate in epidermal healing (FIG. 6, Panels A-C). Instead, Applicant observed that only SG duct cells proliferated (FIG. 6, Panel E). In general, these results support previously published wound healing data in human SGs by Lobitz et al. (Lobitz, W. C., Jr. et al. (1954) J Invest Dermatol. 22:189-198) as well as mouse SGs by Lu et al. (Lu, C. P. et al. (2012) Cell 150:136-150) which showed the contribution of SG duct cells in wound healing. However, since Lu et al. used a different system, Sox9CreER/RosaLacZ or YFP, they could not rule out fully if these contributing cells are in fact coming only from the duct or from the upper acinar part of SGs as well. In addition, they also used the K15CrePR/R26LacZ approach; however, they specifically labeled only lumenal SG cells in the glandular part after early postnatal RU treatment and could not address the question if basal layer myoepithelial cells could respond to the injury. Although Applicant's results are consistent, using later adult postnatal RU applications in K15CrePR/R26LacZ mice allowed Applicant to mark both basal and lumenal layers of the proximal acinar part of SGs. Therefore, Applicant was able to extend this conclusion to both SG layers and make the statement that not only lumenal but basal cells as well from the SG proximal acinar region do not contribute to wound healing of the epidermis. Collectively, previous reports together with Applicant's findings emphasize that both layers of SG cells remain quiescent in the proximal acinar region during wound healing and only SG duct cells were able to proliferate. It still remains to be addressed in the future which duct layer, lumenal or basal, can contribute to wound healing and cells from which layers are able to fully trans-differentiate into epidermal cells long term.

SG LRCs Possess Multipotency and Stem Cells Characteristic In Vivo and has Potential to Trans-Differentiate into the Epidermis Under Prolonged Isolated Wound Healing

As Applicant demonstrated here, K15CrePR/R26LacZ labeled cells in the acinar part of SGs appear to be generally slow cycling, but these cells were able to selectively maintain and participate in the long term homeostasis of the glandular part of SGs (FIG. 3, Panel G). Thus, it suggests that at least part of this SG structure contains cells with SCs characteristic that can maintain this glandular portion. Although K15 labeled cells overlap with SG LRCs in the basal layer of SGs, it also marked cells of the lumenal layer (FIG. 3, Panels C-E) preventing Applicant from determining whether SG LRCs themselves possess stem cells characteristic. In addition, Applicant shows that SG LRCs survive long term and persists for more than 20 weeks of chase illustrating their extreme slow cycling properties. Although the H2BGFP label persists for such a long period of chase, Applicant demonstrates that its intensity slowly diminishes as these SG LRCs slowly divide over time (FIG. 9).

To assess the stem cell properties of SG LRCs in vivo, Applicant had to challenge Applicant's system further since regular wound healing conditions failed to provoke SG acinar cells to contribute to wound healing of the epidermis. Under this special wound condition where Applicant gave SG cells an advantage by preventing wound closure from the surrounding epidermis, Applicant observed that 4 weeks chased H2BGFP labeled SG LRCs can proliferate and trans-differentiate into all epidermal layers (FIG. 7, Panels P, S, T). In this experiment, animals were kept on continuous Doxy treatment allowing us to distinguish SG LRCs from other cells and confirm that SG LRCs themselves are multipotent.

Together, Applicant's results remain in agreement with previously published results on human, mouse and porcine (Miller, S. J. et al. (1998) J Invest Dermatol. 110:13-19; Biedermann, T. et al. (2010) J Invest Dermatol. 130:1996-2009; Lobitz, W. C., Jr. (1956) J Invest Dermatol. 26:247-259, discussion:259-262; Rittie, L. et al. (2013) Am J Pathol. 182:163-171; Lu, C. P. et al. (2012) Cell 150:136-150; Lobitz, W. C., Jr., et al. (1954) J Invest Dermatol. 23:329-344), demonstrating that in general, SG cells can respond and re-epithelialize the skin after wounding. However, for the first time, Applicant shows that under more favorable, isolated, and prolonged wound healing condition, normally quiescent myoepithelial SG LRCs can contribute to and reconstitute a stratified epidermis. Thus, Applicant demonstrates that under favorable conditions, these relatively quiescent SG LRCs can be activated and work as an alternative source of cells confirming that these cells are multipotent with SC characteristics in vivo.

Purification and Characterization of Basal Layer Myoepithelial SG LRCs from the Acinar Sweat Gland Region

To further characterize these SG stem cells (SGSCs), Applicant used the KSTetOff/TreH2BGFP approach to localize and isolate SG myoepithelial LRCs from the proximal acinar part of SGs. Applicant purified SG LRCs and adjacent basal layer cells representing SG non-LRCs, which were predominantly composed of basal layer cells from the acinar and ductal regions. Although all SG LRCs showed myoepithelial characteristics co-expressing SMA and p63, only a fraction of SMA positive cells were LRCs while the remaining majority of SMA positive cells did not display label-retaining characteristics (FIG. 2, Panel F). Thus, in Applicant's experimental setup, Applicant specifically targeted and purified SG LRCs which represented approximately 30 to 40% of all basal SG cells attached to the basement membrane. Consequently, Applicant's SG LRCs purification strategy was different from the strategy recently published by Lu et al., where they purified all myoepithelial cells including those without LRC characteristic as well as lumenal cells in both glandular and ductal parts to identify and characterize distinct unipotent stem cell populations in SGs. They used Abs against α6(CD49) and 01 (CD29) integrins, markers previously published for mammary gland SCs purification (Shackleton, M. et al. (2006) Nature 439:84-88; Stingl, J. et al. (2006) Nature 439:993-997), in conjunction with K14-H2BGFP mice which marked basal cells with high GFP intensity and lumenal or suprabasal layer cells with lower GFP intensity, whereas Applicant focused more selectively on basal myoepithelial cells with LRCs and non-LRCs characteristic. Applicant's microarray data revealed gene expression changes in both basal populations, SG LRCs and SG non-LRCs, when compared to the sole's epidermis. This allowed Applicant to identify the expression of various ion channels important for SG function including the Na⁺/K⁺ ATPase pumps, Atp1a1, Atp1b1, and Atp1b3. In addition, vimentin, which was previously published as a marker of the human SG myoepithelium (Schon, M. et al. (1999) J Cell Sci. 112(Pt 12):1925-1936), was found up-regulated in SG LRCs and SG non-LRCs populations. Interestingly, Applicant found components of BMP signaling including Bmpr1a, Bmpr2, Smad5, Id2, Id3 and Decorin to be up-regulated in the SG when compared to the sole's epidermis (FIG. 5, Panel B) suggesting a quiescent behavior of those populations in vivo. This is consistent with a well-known role of BMP signaling in maintaining quiescence in several adult stem cell populations including hair follicle, hematopoietic, intestinal and neural stem cells (Kobielak, K. et al. (2007) Proc Natl Acad Sci. USA 104:10063-10068; Kandyba, E. et al. (2013) Proc Natl Acad Sci. USA 110(4):1351-1356; Zhang, J. et al. (2003) Nature 425:836-841; Haramis, A. P. et al. (2004) Science 303:1684-1686; He, X. C. et al. (2004) Nat Genet 36:1117-1121; Mira, H. et al. (2010) Cell Stem Cell 7:78-89). Although the direct role of BMP signaling in adult SGSCs has not been investigated yet, Applicant was able to confirm the functional requirement of Bmpr1a during SG formation. In the future, it will be important to develop new genetic tools to specifically address further questions about the function of several newly identified genes in SGs to better understand their role in SG biology both during development and in adult SG homeostasis.

### Plasticity of Sweat Gland Cells to Reconstitute Sweat Glands and Hair Follicles In Vivo

In Applicant's reconstitution assay, Applicant showed that dissociated SG cells were able to generate H2BGFP positive SG structures expressing SG markers K8 and Na⁺/K⁺ ATPase (FIG. 8, Panels D, F, M). Surprisingly, Applicant also observed the formation of a few hair follicles with fully differentiated hair shaft fibers (FIG. 8, Panels A-E′). In addition, both SG and hf appendages were found to coexist in the same region which is physiologically not observed in the mouse sole's epidermis (only SGs) nor back skin (only hfs). Since this finding was unexpected, Applicant tested for possible contamination from surrounding paw's epidermis which could potentially be a source of keratinocytes capable of generating hair under the inductive properties of freshly isolated GFP negative newborn dermal fibroblasts. For Applicant's control, Applicant used unsorted cells isolated from the attached 4 weeks chased sole's epidermis mixed with freshly isolated unmarked dermal fibroblasts and did not observe any H2BGFP positive hf formation. Thus, it is unlikely that the H2BGFP marked hfs resulted from contaminating keratinocytes; however, fewer cells were used in this control experiment when compared to the SG reconstitution assay. Instead, an alternative explanation would be that some SG cells were able to adopt this new hf fate. This observation might suggest that some SG cells still possess plasticity to choose between their final fate decisions dependent on surrounding environmental cues. This raises additional questions to be addressed in the future about signals which promote SGs but not hair fate. Surprisingly in this assay, even when Applicant used freshly isolated dermal fibroblasts from back skin, which normally induce hf formation, the inductive signals from these fibroblasts still induced SG formation in addition to hfs. Thus, it is possible that inductive signals are similar during the development of these appendages. In fact, Plikus et al. published that overexpression of a BMP signaling inhibitor, Noggin, resulted in trans-differentiation of SGs into hfs (Plikus, M. et al. (2004) Am J Pathol. 164:1099-1114). Since Applicant has shown here as well that BMP signaling is critical for SG formation, it will be interesting to address how fine tuning of this pathway might modulate its fate decision in the future.

Taken together, Applicant has explored the role of SG LRCs in SGs and were able to localize them to the basal layer myoepithelial cells of the proximal acinar region. Applicant was able to isolate these SG LRCs which allowed Applicant to further characterize them and determine their gene expression profile. Among these genes, a number of BMP signaling genes were identified and Applicant demonstrated the requirement of this signaling pathway in SG formation. Applicant proposes that SG LRCs are the SC population required for the maintenance and homeostasis of the SG skin appendage. This suggests that at least one distinct stem cell population exists in the proximal acinar region of SGs, which contains relatively quiescent cells contributing only to their own glandular structures during homeostasis and typical wound healing. In fact, Applicant's results are in agreement with previously published observations in human SGs, where SG ducts were completely or partially injured in the dermis (Lobitz, W. C., Jr. (1956) J Invest Dermatol. 26:247-259, discussion:259-262). Interestingly, they observed that the deep portion of SGs maintained their quiescence and survived similar to the acinar part containing SGSCs in Applicant's study. In contrast, the ductal part of human SGs were not able to rebuild the lower acinar part of SGs in vivo, but had instead slowly disappeared (Lobitz, W. C., Jr. (1956) J Invest Dermatol. 26:247-259, discussion:259-262). Moreover, Applicant demonstrated that SG LRCs in the acinar compartment in fact possess multipotency and SCs characteristic in vivo having the potential to trans-differentiate into the epidermis under prolonged isolated wound healing conditions. Finally, Applicant's data also suggest plasticity of SG cells to reconstitute both SGs and hfs in vivo.

The present technology is not limited in scope by the examples, which are intended as illustrations of aspects of the present technology. Any methods that are functionally equivalent are within the scope of the present technology. Various modifications of the present technology in addition to those described herein will become apparent to those skilled in the art from the foregoing description and accompanying figures. Such modifications fall within the scope of the appended claims.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Lengthy table referenced here
US20160075995A1-20160317-T00001
Please refer to the end of the specification for access instructions.

Lengthy table referenced here
US20160075995A1-20160317-T00002
Please refer to the end of the specification for access instructions.

Lengthy table referenced here
US20160075995A1-20160317-T00003
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20160075995A1). An electronic copy of the table will also be available from the USPTO upon request and payment of the fee set forth in 37 CFR 1.19(b)(3).

1. An isolated self-renewable sweat gland stem cell.

2. The isolated sweat gland stem cell of claim 1, wherein the isolated sweat gland stem cell is multipotent.

3. The isolated sweat gland stem cell of claim 1, wherein the isolated sweat gland stem cell is capable of differentiation into at least one cell or tissue type selected from the group of an epidermal cell, a hair follicle or a sweat gland.

4. The isolated sweat gland stem cell of claim 1, wherein the isolated sweat gland stem cell is capable of differentiation into at least two of the cell types.

5. The isolated sweat gland stem cell of claim 1, wherein the isolated sweat gland stem cell can be isolated from the proximal acinar gland region.

6. The isolated sweat gland stem cell of claim 5, wherein the SGSC is not substantially found in the SG ductal region.

7. The isolated sweat gland stem cell of claim 1, wherein the isolated sweat gland stem cell expresses a Bone Morphogenic Protein (BMP) marker.

8. An isolated clonal population of the isolated sweat gland stem cell of claim 1.

9. A substantially homogenous population of isolated SGSC of any claim 1.

10. The isolated sweat gland stem cell of claim 1, further comprising an exogeneous agent or an agent with which the isolated SGSC is not normally associated with in nature.

11. The isolated sweat gland stem cell of claim 10, wherein the agent is one or more of a small molecule, a preservative, a growth factor, a detectable label, an antibody, a non-naturally occurring nucleic acid, a fibroblast, a BMP signaling agonist or a BMP signaling antagonist.

12. A method of culturing a SGSC comprising growing an isolated SGSC of claim 1 or 2, in a growth or differentiation medium that optionally is not a naturally occurring composition.

13. The method of claim 12, further comprising a fibroblast cell.

14. A method for tissue regeneration, comprising administering to the subject an effective amount of the isolated sweat gland stem cell of claim 1, thereby regenerating tissue.

15. A method for treating a burn or wound comprising administering to the subject an effective amount of the isolated sweat gland stem cell of claim 1, thereby treating the burn or wound.

16. The method of claim 15, wherein administration comprises topically or locally applying the cell to the burn or wound area.

17. The method of claim 15, further comprising administering an effective amount of fibroblasts.

18. The method of claim 15, further comprising administering an effective amount of a BMP promoting agent or protein.

19. The method of claim 15, wherein the SGSC are autologous or allogeneic to the subject being treated.

20. A method for identifying an agent that modulates the growth or differentiation of the isolated sweat gland stem cell of any of claim 1, comprising contacting the cell or the population with the agent wherein a change of growth or differentiation of the cell or population indicates that the agent modulates the growth or differentiation of the cell or the population.
